(19) United States
(12) Patent Application Publication
(10) Pub. No.: US 2026/0000511 A1
(43) Pub. Date: Jan. 1, 2026

(54) DELIVERY APPARATUS AND METHODS FOR IMPLANTING PROSTHETIC DEVICES

(71) Applicant: EDWARDS LIFESCIENCES CORPORATION, Irvine, CA (US)

(72) Inventor: John J. Desrosiers, San Clemente, CA (US)

(21) Appl. No.: 19/320,010

(22) Filed: Sep. 5, 2025

Related U.S. Application Data
(63) Continuation of application No. PCT/US2024/018558, filed on Mar. 5, 2024.
(60) Provisional application No. 63/450,300, filed on Mar. 6, 2023.

(51) Int. Cl. A61F 2/24

(52) U.S. Cl. CPC A61F 2/2436

(57) ABSTRACT

A shaft assembly to facilitate delivery of an implant to an implantation location within a patient's anatomy may comprise an outer shaft that is movable between an extended position to encapsulate the implant loaded onto a delivery apparatus and a retracted position to expose the implant for deployment at the implantation location at a distal end of the outer shaft. The outer shaft may be movable from the retracted position to the extended position to encapsulate the implant at least partially within the distal end of the outer shaft, the outer shaft comprising one or more flaring openings at the distal end of the outer shaft.

### CROSS-REFERENCE TO RELATED APPLICATION

This application is a continuation of PCT Patent Application No. PCT/US2024/018558 filed on Mar. 5, 2024, which claims priority to U.S. Provisional Patent Application No. 63/450,300, filed Mar. 6, 2023, each of these applications being incorporated by reference herein in its entirety.

### FIELD

The present disclosure relates generally to delivery apparatus and methods for implanting prosthetic devices and more particularly to delivery apparatus and method for implanting support structures and/or prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable.

In one specific example, a prosthetic valve can be mounted in a crimped state on the distal end of a delivery apparatus and advanced through the patient's vasculature (e.g., through a femoral artery and the aorta) until the prosthetic valve reaches the implantation location in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

In some cases, it may not be possible to secure the prosthetic valve to the native valve annulus, for example, if the native valve annulus is too large or if the geometry of the native valve is too complex to allow secure implantation of the valve. One approach in these cases is to first deploy a docking station at the implantation location and then install the prosthetic valve in the docking station. The docking station can be selected to provide the necessary interface to anchor the prosthetic valve within the native valve annulus. Desirably, the docking station can be delivered to the implantation location with a minimally invasive procedure, which would allow the docking station to be deployed within the same procedure used to deliver the prosthetic valve.

### SUMMARY

Disclosed herein are examples of a delivery apparatus that can be used to deliver a prosthetic implant, such as a docking station, to an implantation location within a patient's body. The delivery apparatus includes a handle and a shaft assembly coupled to the handle. The shaft assembly includes an outer shaft and an inner shaft extending through a lumen of the outer shaft. A carriage within the handle is coupled to the outer shaft and movable relative to the handle to displace the outer shaft axially and relative to the handle. Movement of the carriage can displace the outer shaft between an extended position to capture the prosthetic implant and a retracted position to expose the prosthetic implant. In some examples, the carriage includes a stepdown shoulder that together with a proximal end of the outer shaft form a gland. A seal member may be disposed within the gland to seal between the carriage and the shaft assembly. The stepdown shoulder in the carriage allows the carriage to be molded as a single piece, simplifying both manufacture and assembly of the delivery apparatus. In some examples, the inner shaft includes one or more fluid ports that fluidly connect a lumen of the inner shaft to the lumen of the outer shaft, allowing both lumens to be flushed from a single injection port. In some examples, a frame connector is provided that couples the implant device to the inner shaft. The frame connector includes a recess that is configured to receive a connector tab on the implant device. The recess has at least one undercut wall that translates tensile force applied to the connector tab to radial force acting on the connector tab, which can improve retention characteristics of the prosthetic implant prior to deploying the prosthetic implant at the implantation location and particularly during recapture of the prosthetic implant.

In one representative example, a delivery apparatus comprises a handle body, a carriage member, an outer shaft, an inner shaft, and a seal member. The handle body includes a proximal end, a distal end, a longitudinal axis extending between the proximal end and the distal end, and a cavity disposed between the proximal end and the distal end. The carriage member is disposed within the cavity and axially movable relative to the handle body in a direction parallel to the longitudinal axis of the handle body. The carriage member has an internal surface defining an internal bore and a gland shoulder integrally formed with the internal surface and defining a stepdown transition in the internal bore. The outer shaft includes a proximal end positioned within the internal bore of the carriage member and in opposing relation to the gland shoulder. The proximal end of the outer shaft, the gland shoulder of the carriage member, and a portion of the internal surface of the carriage member adjacent to the stepdown transition define an annular groove. The inner shaft extends through a lumen of the outer shaft and is fixed relative to the handle body. The seal member is disposed around the inner shaft and within the annular groove and is positioned to form a seal between the carriage member and the inner shaft and at the proximal end of the outer shaft.

In another representative example, a delivery assembly comprises the preceding delivery apparatus and an expandable docking station releasably coupled to the delivery apparatus, wherein the expandable docking station is configured to receive a prosthetic heart valve.

In another representative example, a method comprises inserting a distal end of the preceding delivery assembly into a vasculature of a patient, advancing the delivery assembly through the vasculature of the patient to position the expandable docking station at a select implantation location, and moving the carriage member relative to the handle to actuate the outer shaft to unsheathe and release the expandable docking station from the delivery apparatus.

In another representative example, a handle for a prosthetic implant delivery apparatus comprises a handle body and a carriage member. The handle body includes a longitudinal axis and a cavity extending along the longitudinal axis. The carriage member is disposed within the cavity and axially movable relative to the longitudinal axis of the handle body. The carriage member comprises a carriage body having an internal surface defining an internal bore and a gland shoulder integrally formed with the internal surface and defining a stepdown transition in the internal bore. The gland shoulder and a portion of the internal surface adjacent to the stepdown transition form a portion of an annular groove configured to receive a seal member.

In another representative example, a carriage for a prosthetic implant delivery apparatus comprises a one-piece molded body having an internal surface defining an internal bore and a gland shoulder integrally formed with the internal surface and defining a stepdown transition in the internal bore. The gland shoulder and a portion of the internal surface adjacent to the stepdown transition form a portion of an annular groove configured for receiving a seal member.

In another representative example, a method of forming a component of a prosthetic implant delivery apparatus comprises securing a core pin within a mold cavity and injecting a thermoplastic material into the mold cavity to form a molded body having an internal surface defining an internal bore and a gland shoulder integrally formed with the internal surface and defining a first stepdown transition in the internal bore.

In another representative example, a method of forming a component of a prosthetic implant delivery apparatus comprises securing a core pin within a mold cavity and injecting a thermoplastic material into the mold cavity to form a mold body having an internal surface defining an internal bore, a gland shoulder integrally formed with the internal surface and defining a stepdown transition in the internal bore, and a locating shoulder integrally formed with the internal surface and axially displaced from the gland shoulder.

In another representative example, a delivery apparatus comprises a handle body, an outer shaft, an inner shaft, and an injection port. The handle body includes a longitudinal axis and a cavity extending along the longitudinal axis. The outer shaft includes a proximal end positioned within the cavity. The outer shaft has a first lumen. The inner shaft extends through the first lumen of the outer shaft. The inner shaft has a second lumen and one or more fluid ports that fluidly connect the second lumen to the first lumen. The injection port is fluidly connected to the second lumen of the inner shaft, and both the first and second lumens are flushable with a fluid through the injection port.

In another representative example, a delivery assembly comprises the preceding delivery apparatus and an expandable docking station for an expandable valve releasably coupled to the delivery apparatus.

In another representative example, a method comprises inserting a distal end of the preceding delivery assembly into a vasculature of a patient, advancing the distal end of the delivery assembly through the vasculature of the patient to position the expandable docking station at a select implantation location, and moving the carriage member relative to the handle to actuate the outer shaft to unsheathe and release the expandable docking station from the delivery apparatus.

In another representative example, a method comprises providing a reinforced tube comprising an inner layer, a reinforcement layer disposed over the inner layer, and an outer layer disposed over the reinforcement layer. The method includes ablating the reinforced tube at one or more locations to form one or more fluid ports in the reinforced tube.

In another representative example, a method comprises disposing a cover tube having one or more windows over a reinforced tube and ablating the reinforced tube at one or more locations exposed through the one or more windows to form one or more fluid ports in the reinforced tube.

In another representative example, a shaft assembly for a prosthetic implant delivery apparatus comprises an outer shaft having a first lumen and an inner shaft extending through the first lumen. The inner shaft comprises a reinforced tube having a second lumen and one or more fluid ports that fluidly connect the second lumen to the first lumen. The inner shaft further includes a cover tube disposed over the reinforced tube. The cover tube has one or more windows positioned to expose the one or more fluid ports to the first lumen.

In another representative example, a delivery apparatus comprises an elongate shaft and a frame connector. The elongate shaft has a proximal end portion and a distal end portion. The proximal end portion is configured to be disposed outside of a patient's body during a delivery procedure, and the distal end portion is configured to be disposed inside a patient's body during the delivery procedure. The frame connector is coupled to the distal end portion of the elongate shaft and configured for releasably coupling a prosthetic implant to the delivery apparatus. The frame connector comprises a connector body having an exterior with an exterior surface and a recess. The recess comprises a first slot portion having a first width, a second slot portion having a second width that is larger than the first width, a recess floor, and opposed first and second side walls extending from the recess floor to the exterior surface and connected to the first slot portion and the second slot portion. At least a first portion of each of the first and second side walls connected to the second slot portion includes an undercut from the exterior surface to the recess floor.

In another representative example, a frame connector for a prosthetic implant delivery apparatus comprises a connector body having an exterior with an exterior surface, a recessed surface, and opposed first and second side walls. The recessed surface is spaced radially inwardly relative to the exterior surface and comprises a first slot portion having a first width and a second slot portion having a second width that is larger than the first width. The opposed first and second side walls extend radially from the recessed surface to the exterior surface and are connected to the first slot portion and the second slot portion. At least a first portion of each of the first and second side walls connected to the second slot portion forms an angle relative to the recessed surface within a range of 75-89.9 degrees.

In another representative example, a delivery assembly comprises a self-expandable docking station and a frame connector. The self-expandable docking station comprises at least one connector tab having a flared portion. The frame connector comprises a connector body having at least one recess to receive and retain the at least one connector tab. The at least one recess comprises a slot portion to receive the flared portion, a recess floor, and opposed first and second side walls connected to the slot portion and the recess floor. At least a portion of each of the first and second side walls forms an angle relative to the recess floor within a range of 75-89.9 degrees.

In another representative example, a shaft assembly to facilitate delivery of an implant to an implantation location within a patient's anatomy may comprise an outer shaft that is movable between an extended position to encapsulate the implant loaded onto a delivery apparatus and a retracted position to expose the implant for deployment at the implantation location at a distal end of the outer shaft. The outer shaft may be movable from the retracted position to the extended position to encapsulate the implant at least partially within the distal end of the outer shaft, the outer shaft comprising one or more flaring openings at the distal end of the outer shaft.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is an elevation view of a portion of a frame of a docking station in a radially-expanded state.

FIG. 2 is a perspective view of the frame of FIG. 1 in a radially-compressed state.

FIG. 3 is a perspective view of a docking station including the frame of FIG. 1.

FIG. 4 is a cut-away view of the docking station of FIG. 3 deployed at an implantation location within a patient's anatomy, which is depicted schematically in cross-section, and with a prosthetic heart valve deployed therein.

FIG. 5A is a perspective view of a delivery apparatus for deploying a docking station.

FIG. 5B illustrates the docking station of FIG. 3 disposed around a distal portion of the delivery apparatus of FIG. 5A.

FIG. 6A is an elevation view of a distal portion of the delivery apparatus of FIG. 5A with an outer shaft of the delivery apparatus in a retracted position.

FIG. 6B is an elevation view of a distal portion of the delivery apparatus of FIG. 5A with an outer shaft of the delivery apparatus in an extended position and cut away to show an encapsulated docking station.

FIGS. 6C-6F illustrate stages in deployment of the docking station of FIG. 3 from the delivery apparatus of FIG. 5A.

FIG. 7A is a perspective view of a handle portion of the delivery apparatus illustrated in FIG. 5A.

FIGS. 7B and 7C are perspective views of the handle portion of FIG. 7A with a portion of the handle cut away to show various internal components.

FIGS. 8A and 8B are perspective views of a carriage member of the handle portion of FIG. 7A.

FIG. 8C is a cross-sectional view of the carriage member of FIGS. 8A and 8B.

FIG. 9 is a cross-sectional view of a head portion of the carriage member of FIGS. 8A and 8B.

FIG. 10 is a cross-sectional view of the carriage member of FIGS. 8A and 8B with a proximal portion of a shaft assembly extending through the carriage member.

FIG. 11A is a cross-sectional view of the handle portion of FIG. 7A, taken along a plane intersecting line 11A-11A as depicted in FIG. 7A.

FIG. 11B is a cross-sectional view of the handle portion of FIG. 7A, taken along line 11B-11B as depicted in FIG. 11A.

FIG. 12A is an elevation view of a proximal portion of the shaft assembly coupled to the handle portion of FIG. 7A with a portion of the shaft assembly cut away to show fluid ports in an inner shaft of the shaft assembly.

FIG. 12B is a cross-sectional view of a portion of the inner shaft of the shaft assembly illustrated in FIG. 12A.

FIG. 12C is an enlarged view of the region 12C as depicted in FIG. 12A.

FIGS. 13A and 13B are elevation views of a frame connector.

FIG. 14 is a perspective view of the frame connector of FIGS. 13A and 13B with a cut-away plane taken along line 14-14 as depicted in FIG. 13A.

FIG. 15 illustrates the frame connector of FIGS. 13A and 13B with a connector tab of a docking station retained in a recess of the frame connector.

FIG. 16A is a perspective view of the frame connector of FIGS. 13A and 13B with a cut-away plane taken along line 16A-16A as depicted in FIG. 13A.

FIG. 16B is a cross-sectional view of the frame connector of FIGS. 13A and 13B at the cut-away plane shown in FIG. 16A.

FIG. 17A is a perspective view of the frame connector of FIGS. 13A and 13B with a cut-away plane taken along line 17A-17A as depicted in FIG. 13A.

FIG. 17B is a cross-sectional view of the frame connector of FIGS. 13A and 13B at the cut-away plane shown in FIG. 17A.

FIG. 18 is a cross-sectional view of a distal portion of a delivery apparatus illustrating the frame connector of FIGS. 13A and 13B connected to an inner shaft of the shaft assembly of FIGS. 5A and 5B.

FIG. 19 is an elevation view of a distal portion of the delivery apparatus of FIG. 5A with an outer shaft of the delivery apparatus in an extended position and cut away to show a docking station restrained by the outer shaft and the frame connector of FIGS. 13A and 13B.

FIG. 20 is a rotated view of the distal portion of the delivery apparatus depicted in FIG. 19 with the frame connector and outer shaft cut away to show engagement with connector tabs of a docking station.

FIG. 21 illustrates radial deflection of the connector tabs of the docking station of FIGS. 19 and 20 in response to axial tension applied to the connector tabs.

FIGS. 22A-22F illustrate operation of a delivery apparatus with an outer shaft that includes one or more flaring openings to facilitate retraction of an implant.

FIGS. 23A-23D illustrate perspective views of various embodiments of outer shafts that include one or more flaring openings.

FIGS. 24A-24D illustrate longitudinal views of various configurations of embodiments of an outer shaft with multiple flaring openings.

FIGS. 25A-25G illustrate front views of various embodiments of flaring openings in an outer shaft.

FIGS. 26A-26H illustrate front views of various profiles of phalanges created by flaring openings in an outer shaft or profiles of the flaring openings themselves at a distal end of an outer shaft.

FIGS. 27A-27D illustrate various views of an outer shaft that includes webbing.

### DETAILED DESCRIPTION

### General Considerations

For the purposes of this description, certain specific details are set forth herein in order to provide a thorough understanding of disclosed examples. In some cases, as will be recognized by one skilled in the art, the disclosed examples may be practiced without one or more of these specific details, or may be practiced with other methods, structures, and materials not specifically disclosed herein. In some instances, well-known structures and/or processes associated with prosthetic valves and delivery apparatuses have been omitted to avoid obscuring novel and non-obvious aspects of the disclosed examples.

The disclosed technology is described via examples and implementations. All the examples and implementations described herein and shown in the drawings may be combined without any restrictions to form any number of combinations, unless the context clearly dictates otherwise, such as if the proposed combination involves elements that are incompatible or mutually exclusive. The sequential order of the acts in any process described herein may be rearranged, unless the context clearly dictates otherwise, such as if one act requires the result of another act as input.

In the interest of conciseness, and for the sake of continuity in the description, same or similar reference characters may be used for same or similar elements in different figures, and description of an element in one figure will be deemed to carry over when the element appears in other figures with the same or similar reference character. In some cases, the term “corresponding to” may be used to describe correspondence between elements of different figures. In an example usage, when an element in a first figure is described as corresponding to another element in a second figure, the element in the first figure is deemed to have the characteristics of the other element in the second figure, and vice versa, unless stated otherwise.

The word “comprise” and derivatives thereof, such as “comprises” and “comprising”, are to be construed in an open, inclusive sense, that is, as “including, but not limited to”. The singular forms “a”, “an”, “at least one”, and “the” include plural referents, unless the context dictates otherwise. The term “and/or”, when used between the last two elements of a list of elements, means any one or more of the listed elements. The term “or” is generally employed in its broadest sense, that is, as meaning “and/or”, unless the context clearly dictates otherwise.

The term “coupled” without a qualifier generally means physically coupled or linked and does not exclude the presence of intermediate elements between the coupled elements absent specific contrary language. The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

### Introduction to the Disclosed Technology

This disclosure describes a plurality of delivery apparatus that can be used to deliver prosthetic implants such as docking stations and/or prosthetic heart valves to an implantation location within a patient's anatomy. The delivery apparatus includes a shaft assembly coupled to a handle, which controls operations of the delivery apparatus. A prosthetic implant can be encapsulated within a distal end portion of one of the shafts of the shaft assembly for delivery to the implantation location.

The shaft assembly includes an outer shaft that is movable between an extended position to encapsulate a prosthetic implant loaded onto the delivery apparatus and a retracted position to expose the prosthetic implant for deployment at the implantation location. A carriage member is included in the handle to move the outer shaft between the retracted and extended positions. The shaft assembly includes an inner shaft that extends through the lumen of the outer shaft.

In certain examples, the outer shaft includes one or more flaring openings at a distal end of the outer shaft. The flaring openings may include slits or other-shaped openings that facilitate an expansion of the effective outer circumference of the distal end of the outer shaft. For example, the flaring openings may facilitate one or more portions of the distal end of the outer shaft to flare radially outward to facilitate extension of the outer shaft back over the implant to return the implant back into the outer shaft. By providing the flaring openings, an angle of interaction between a given surface of the implant and the inner surface of the outer shaft may be adjusted such that less force is required to extend the outer shaft over the implant to re-encapsulate the implant within the outer shaft.

In certain examples, the carriage member and the outer shaft form a gland or annular groove to hold a seal member. In certain examples, the inner shaft includes one or more fluid ports that together with the seal member disposed within the carriage member allow the inner shaft and the outer shaft to be flushed with fluid from a single injection port.

In certain examples, the inner shaft can carry a frame connector having one or more recesses to receive one or more connector tabs of the prosthetic implant and thereby axially restrain the prosthetic implant. In certain examples, the recesses have undercut walls that translate tensile force applied to the connector tabs to radial force acting on the connector tabs, which can help maintain engagement of the connector tabs with the recesses during recompression and/or retrieval of the prosthetic implant.

### Examples of the Disclosed Technology

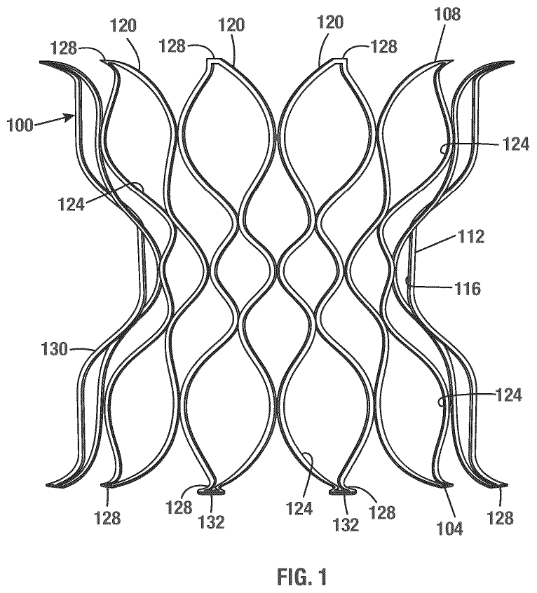

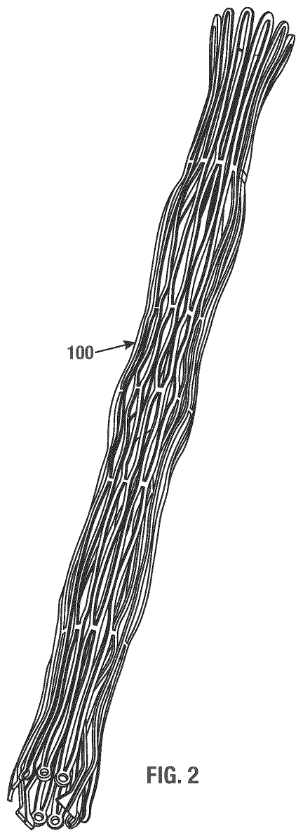

Turning now to the drawings, FIG. 1 illustrates an exemplary implementation of a frame 100 (or stent) that can form a body of a docking station. The frame 100 has a first end 104 and a second end 108. In some examples, the first end 104 can be an inflow end, and the second end 108 can be an outflow end. In other examples, the first end 104 can be an outflow end, and the second end 108 can be an inflow end. The terms “inflow” and “outflow” are related to the normal direction of blood flow (e.g., antegrade blood flow) through the frame. In the unconstrained, expanded state of the frame 100 shown in FIG. 1, a relatively narrower portion (or waist) 112 of the frame 100 between the first end 104 and the second end 108 forms a valve seat 116. The frame 100 can be compressed (as illustrated in FIG. 2) for delivery to an implantation location by a delivery apparatus.

Although the docking stations, delivery apparatus, prosthetic heart valves, and/or methods are described herein with respect to a particular implantation location (e.g., a pulmonary valve) and/or a particular delivery approach (e.g., transfemoral), the device and methods disclosed herein can be adapted to various other implantation locations (e.g., an aortic valve, a mitral valve, and a tricuspid valve) and/or delivery approaches (e.g., transapical, transseptal, etc.).

In the example illustrated by FIG. 1, the frame 100 includes a plurality of struts 120 arranged to form cells 124. The ends of the struts 120 form apices 128 at the ends of the frame 100. One or more of the apices 128 can include a connector tab 132. The portions of the struts 120 between the apices 128 and the valve seat 116 (or the waist 112) form a sealing portion 130 of the frame 100. In the unconstrained, expanded state of the frame 100 illustrated in FIG. 1, the apices 128 extend generally radially outward and are radially outward of the valve seat 116.

The frame 100 can be made of a highly resilient or compliant material to accommodate large variations in the anatomy. For example, the frame 100 can be made of a flexible metal, metal alloy, polymer, or an open cell foam. An example of a highly resilient metal is Nitinol, which is a metal alloy of nickel and titanium, but other metals and high resilient or compliant non-metal materials can be used. The frame 100 can be self-expanding, manually expandable (e.g., expandable via a balloon), or mechanically expandable. A self-expanding frame can be made of a shape memory material, such as, for example, Nitinol. In this manner, the frame can be radially compressed as depicted in FIG. 2 (e.g., via a crimping device) and can radially expand to the configuration depicted in FIG. 1.

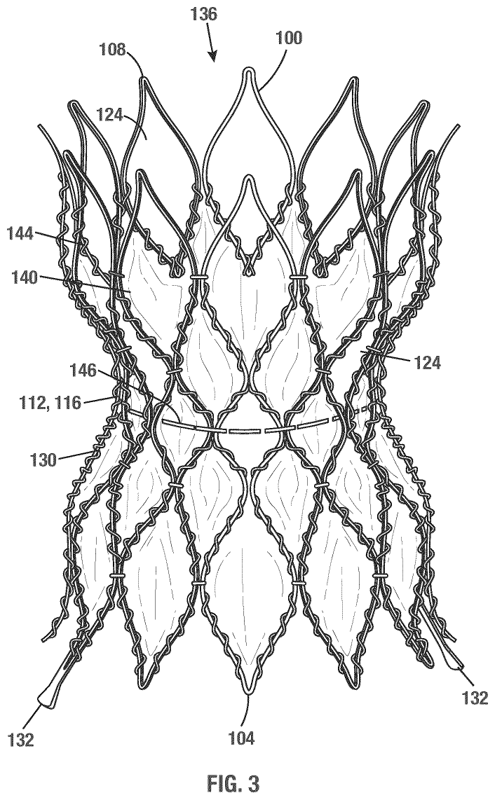

FIG. 3 illustrates an exemplary docking station 136 including the frame 100 and an impermeable material 140 disposed within the frame. The impermeable material 140 is attached to the frame 100 (e.g., by sutures 144). In the example illustrated by FIG. 3, the impermeable material 140 covers at least the cells 124 in the sealing portion 130 of the frame 100. The seal formed by the impermeable material 140 at the sealing portion 130 can help funnel blood flowing into the docking station 136 from the proximal inflow end 104 to the valve seat 116 (and the valve once installed in the valve seat). One or more rows of cells 124 proximate to the distal outflow end 108 can be open.

The impermeable material 140 can be a fabric that is impermeable to blood. A variety of biocompatible materials can be used as the impermeable material 140, such as, for example, foam or a fabric that is treated with a coating that is impermeable to blood, a polyester material, or a processed biological material, such as pericardium. In one particular example, the impermeable material 140 can be polyethylene terephthalate (PET).

The docking station 136 may include a band 146 that extends around the waist 112 (or that is integral to the waist) of the frame 100. The band 146 can constrain expansion of the valve seat 116 to a specific diameter in the deployed state to enable the valve seat 116 to support a specific valve size. The band 146 can take on a wide variety of different forms and can be made of a wide variety of different materials. For example, the band 146 can be made of PET, one or more sutures, fabric, metal, polymer, a biocompatible tape, or other relatively nonexpanding materials known in the art and that can maintain the shape of the valve seat 116.

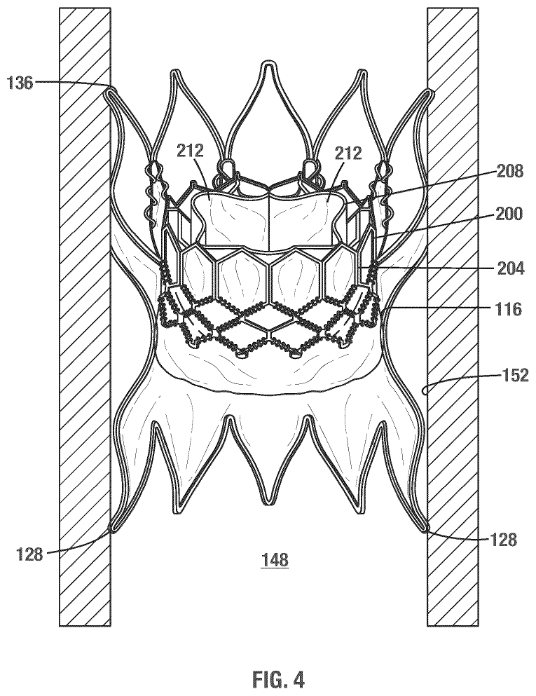

FIG. 4 illustrates the docking station 136 in a deployed state within a native valve annulus 148. As can be seen, the frame 100 of the docking station 136 is in an expanded condition, with the end portions of the frame pressed against the inner surface 152 of the native valve annulus. The band 146 (shown in FIG. 3) can maintain the valve seat 116 at a constant or substantially constant diameter in the expanded condition of the frame 100. FIG. 4 also shows a prosthetic valve 200 deployed within the docking station 136 and engaged with the valve seat 116 of the docking station 136. The prosthetic valve 200 can be implanted by first deploying the docking station 136 at the implantation location and then installing the prosthetic valve within the docking station.

The prosthetic valve 200 can be configured to replace a native heart valve (e.g., aortic, mitral, pulmonary, and/or tricuspid valves). In one example, the prosthetic valve 200 can include a frame 204 and a valvular structure 208 disposed within and attached to the frame 204. The valvular structure 208 can include one or more leaflets 212 that cycle between open and closed states during the diastolic and systolic phases of the heart. The frame 204 can be made of the frame materials described for the frame 100 of the docking station 136. The leaflets 212 can be made in whole or in part from pericardial tissue (e.g., bovine pericardial tissue), biocompatible synthetic materials, or various other suitable natural or synthetic materials known in the art.

The docking station 136 is not limited to use with the particular example of the prosthetic valve 200 illustrated in FIG. 4. For example, mechanically expandable prosthetic valves such as described in U.S. Patent Publication Nos. 2018/0153689 and 2019/0060057; U.S. Patent Application No. 62/869,948; and International Application No. PCT/US2019/056865, the relevant disclosures of which are incorporated by reference herein, may be installed in the docking station 136.

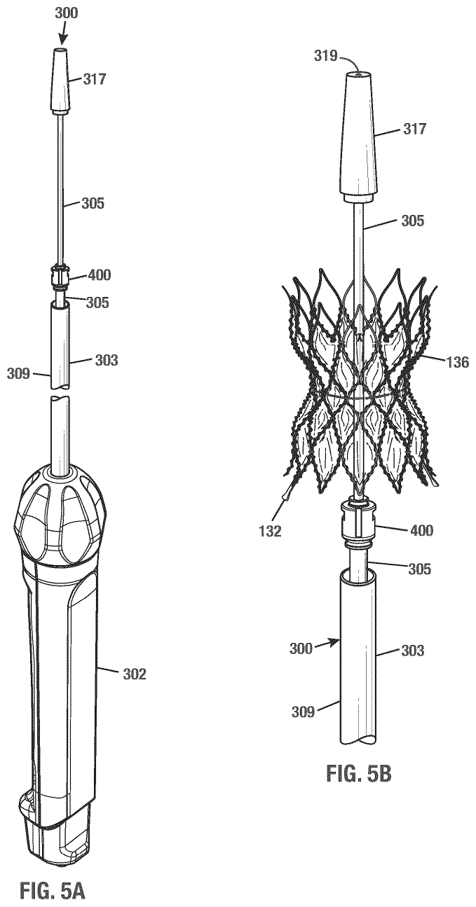

FIG. 5A illustrates an exemplary delivery apparatus 300 that can be used to deliver the docking station to an implantation location. The delivery apparatus 300 generally includes a handle 302 and a shaft assembly 303 coupled to the handle 302 and extending distally from the handle 302. The shaft assembly 303 includes an inner shaft 305 and an outer shaft 309. The inner shaft 305 extends through a lumen of the outer shaft 309.

In the example illustrated by FIG. 5A, a frame connector 400 is coupled to the inner shaft 305. The docking station 136 can be disposed around a portion of the inner shaft 305 extending distally from the frame connector 400, as shown in FIG. 5B. In one example, the frame connector 400 includes one or more recesses that can receive one or more connector tabs 132 at the proximal end of the docking station 136 and thereby axially restrain the docking station 136.

A nosecone 317 can be attached to a distal end of the inner shaft 305. The nosecone 317 includes a central opening 319 for receiving a guidewire. As such, a proximal end of the guidewire can be inserted into the central opening 319 and through the inner shaft 305, and a distal end portion of the delivery apparatus 300 can be advanced over the guidewire through a patient's vasculature and to an implantation location. The guidewire can pass through the nosecone 317 into the inner shaft 305 during advancing of the delivery apparatus through a patient's vasculature.

The handle 302 can be operated to move the outer shaft 309 relative to the inner shaft 305, generally between an extended position and a retracted position. The handle 302 can be actuated to slide the outer shaft 309 over the frame connector 400 and over any docking station coupled to the frame connector 400 to encapsulate the docking station within the outer shaft 309. As the outer shaft 309 slides over the docking station 136, the outer shaft 309 can compress the docking station 136 such that the docking station is encapsulated within the outer shaft 309 in the compressed state. In the fully extended position, a distal end of the outer shaft 309 can abut a proximal end of the nosecone 317 such that there are no gaps in the delivery assembly. Additionally (or alternatively), a crimping device can be used to radially compress the docking station such that it can be inserted into the outer shaft of the delivery apparatus.

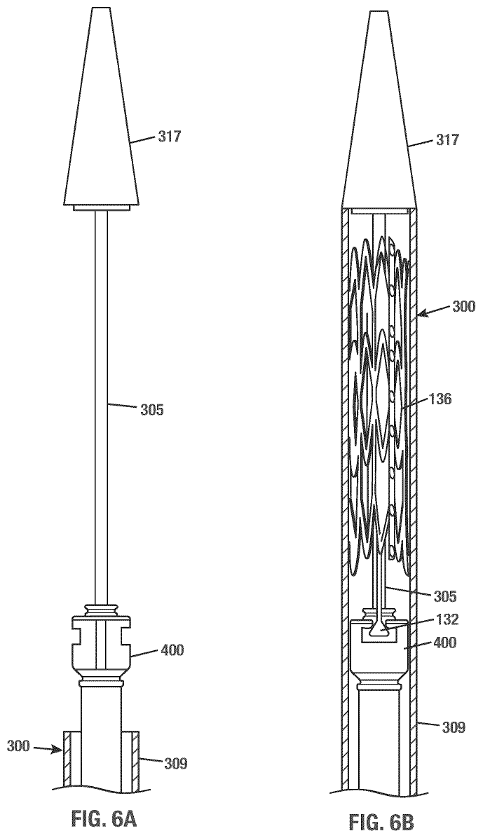

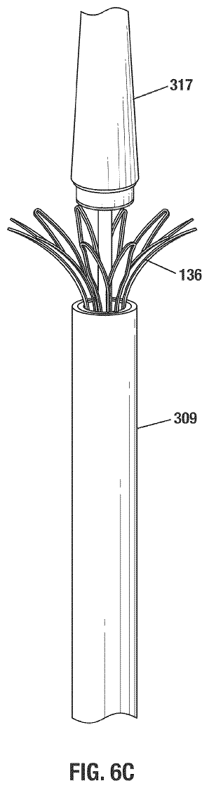

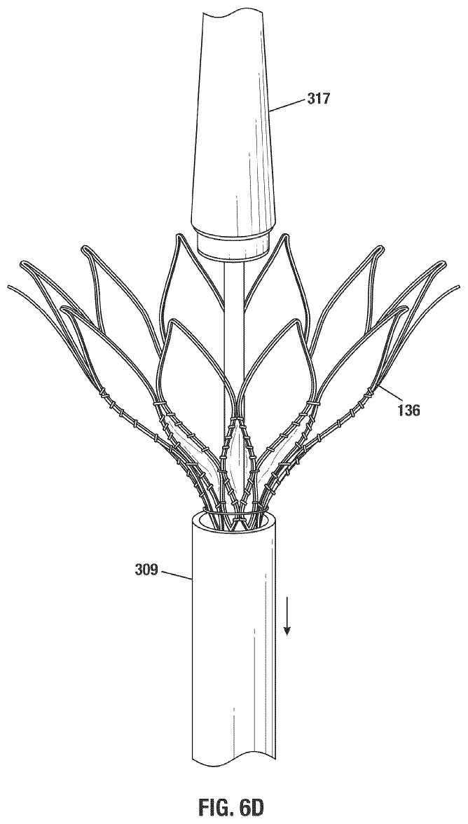

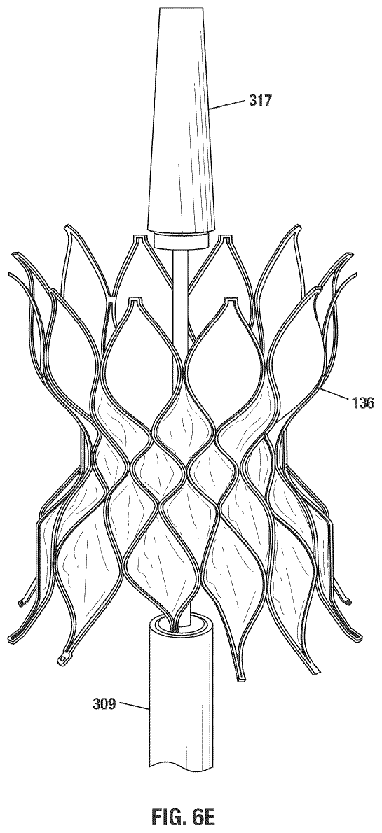

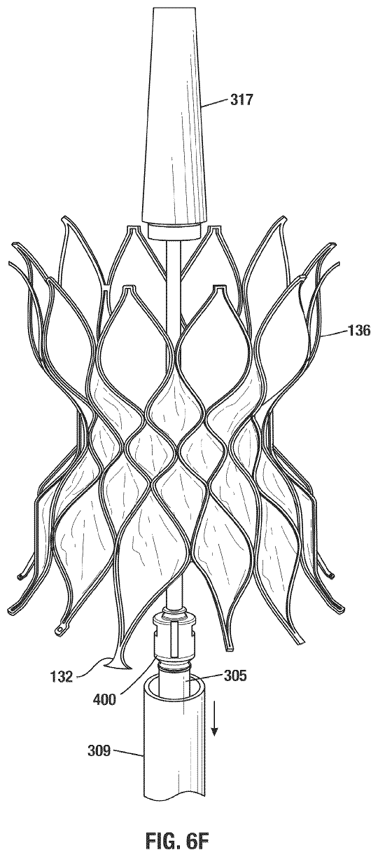

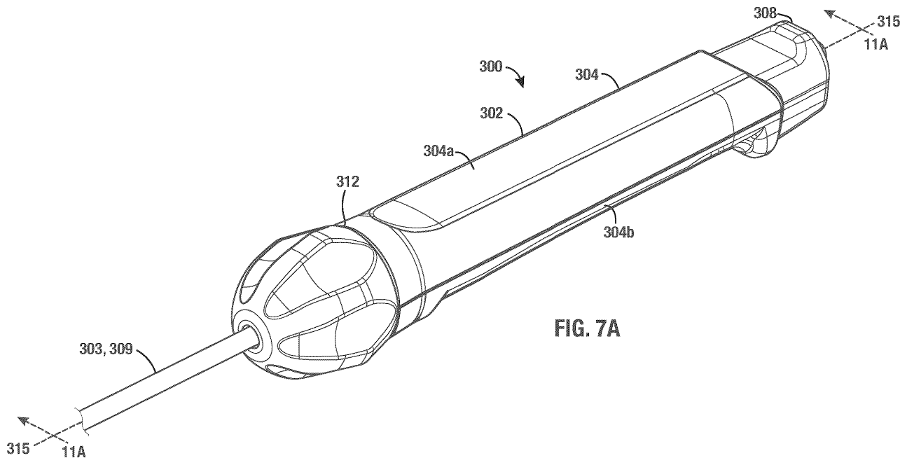

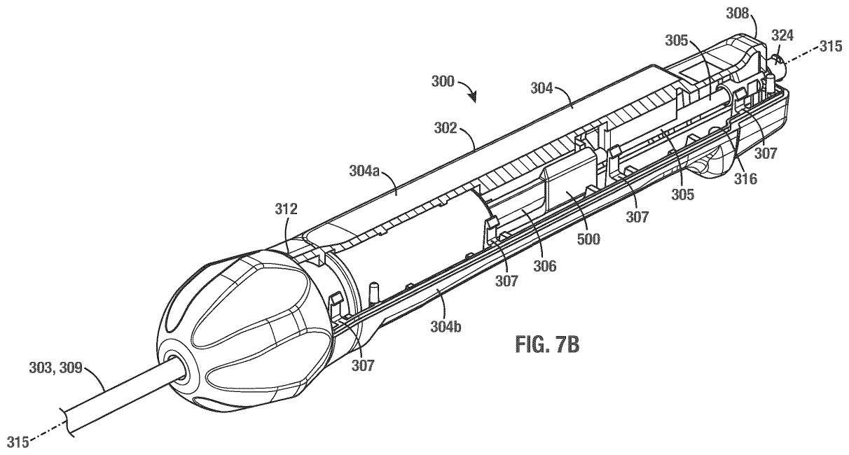

FIGS. 6A-7C illustrate a method of deploying a docking station at an implantation location within an anatomy. For purposes of illustration, the patient's anatomy is omitted. In FIG. 6A, the method includes retracting the outer shaft 309 by the handle of the delivery apparatus to allow loading of the docking station 136 onto the inner shaft 305. In FIG. 6B, the method includes disposing the docking station 136 around the inner shaft 305 and engaging each of the connector tabs 132 of the docking station 136 with the frame connector 400. The method also includes positioning the outer shaft 309 over the docking station such that the docking station is encapsulated therein. This can be accomplished by manipulating the handle of the delivery apparatus. As shown in FIG. 6B, the distal end of the outer shaft 309 abuts the proximal end of the nosecone 317. The method includes inserting the delivery apparatus, from the nosecone 317 end, into a patient's vasculature and advancing the delivery apparatus through the patient's vasculature to the implantation location.

At the implantation location, the method includes retracting the outer shaft 309 by the handle of the delivery apparatus to expose the docking station 136. FIGS. 6C-6F show different stages of retracting the outer shaft 309. As can be seen, in cases where the docking station 136 is self-expanding, the docking station 136 gradually emerges from the outer shaft 309 and gradually expands from the compressed state as the outer shaft 309 is retracted. As the docking station 136 expands it engages with the anatomy. When the outer shaft 309 is sufficiently retracted, the connector tabs 132 disengage from the frame connector 400. Once the docking station 136 is disengaged from the frame connector 400, the docking station 136 can finalize radial expansion to fully engage the anatomy.

FIGS. 7A-7C illustrate an exemplary implementation of the handle 302 of the delivery apparatus. The handle 302 includes a handle body 304 and a deployment mechanism 306 coupled to and partially disposed within the handle body. The handle body 304 includes a proximal end 308, a distal end 312, and a cavity 316 extending from the proximal end 308 to the distal end 312. The handle 302 includes a longitudinal axis 315 extending from the proximal end 308 to the distal end 312, and extending distally through shaft assembly 303. The longitudinal axis 315 defines the axial direction of the handle.

The handle body 304 can be a single piece body with the cavity 316. Alternatively, the handle body 304 can have two body pieces 304a, 304b that can be assembled together to form the cavity 316. For example, the first body piece 304b may have snap hooks 307 that snap into complementary recesses in the second body piece 304a.

The deployment mechanism 306 of the handle 302 includes a carriage member 500 and a drive member 320. The carriage member 500 is disposed within the cavity 316 and movable relative to the handle body 304 in the axial direction. The drive member 320 engages with the carriage member 500 and is movable (e.g., rotatable) relative to the handle body 304 to adjust the axial position of the carriage member 500 relative to the handle body 304.

Proximal portions of the shafts 305, 309 are inserted into the cavity of the handle body 304. A proximal end portion of the outer shaft 309 of the shaft assembly 303 can be coupled to the carriage member 500 (e.g., by fasteners, adhesive, and/or other means for coupling) such that movement of the carriage member 500 relative to the handle body 304 causes movement of the outer shaft 309 between the extended and retracted positions.

A proximal portion of the inner shaft 305 extends through a lumen 313 of the outer shaft 309 into a proximal portion of the cavity 316 and is coupled to the handle body 304. The inner shaft 305 can be fixed relative to the handle body 304 such that the inner shaft 305 is stationary while the outer shaft 309 moves relative to the handle body 304.

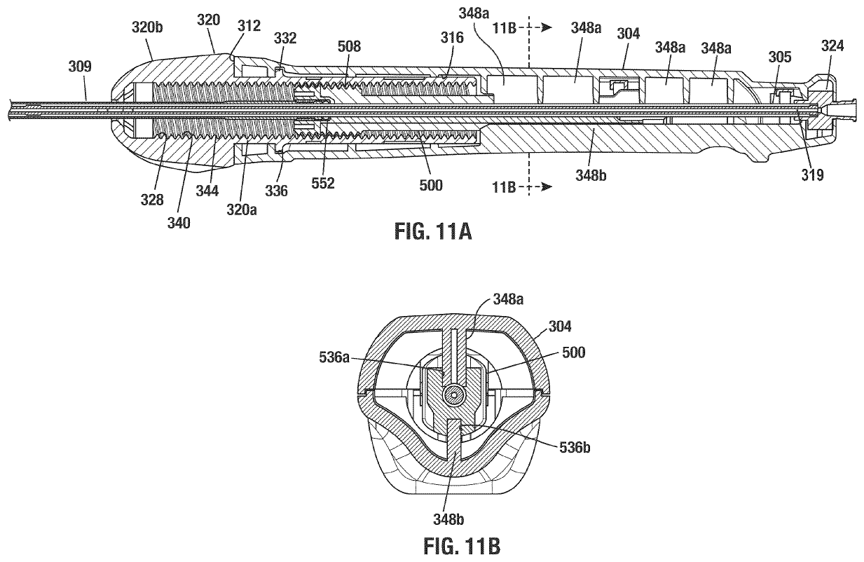

In the example illustrated by FIGS. 7A-7C, an injection port 324 is mounted at an opening at the proximal end 308 of the handle body 304. The injection port 324 can be, for example, a Luer fitting. A proximal end of the inner shaft 305 can be inserted into the injector port 324 (shown in FIG. 11A) and secured to the injection port 324 (e.g., by bonding). In some cases, the attachment of the inner shaft 305 to the injection port 324 can serve the purpose of fixing the inner shaft 305 relative to the handle body 304.

The injection port 324 can be used to inject flushing fluid, such as saline, into the lumen 319 (shown in FIG. 11A) of the inner shaft 305. In some cases, the inner shaft 305 can include one or more fluid ports 311 through which the injected fluid exits the inner shaft 305 and enters the lumen 313 of the outer shaft 309, thereby allowing flushing of the lumens of the inner shaft 305 and outer shaft 309 from a single injection port.

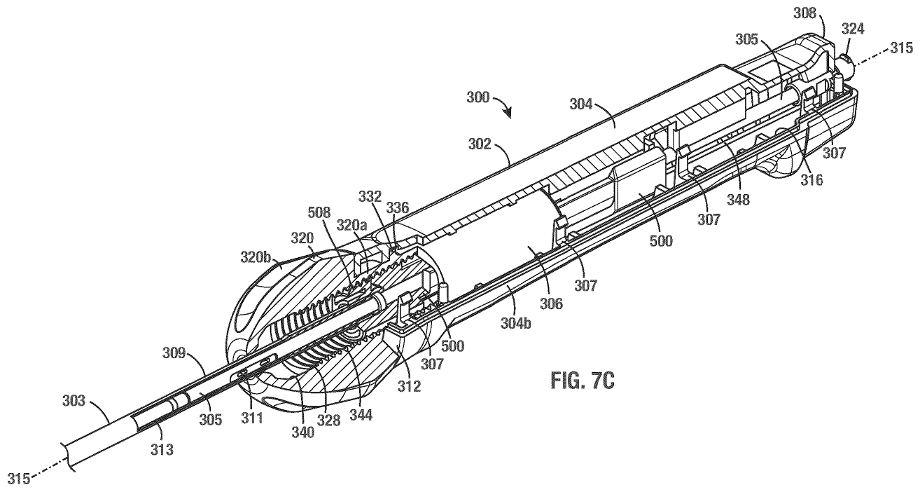

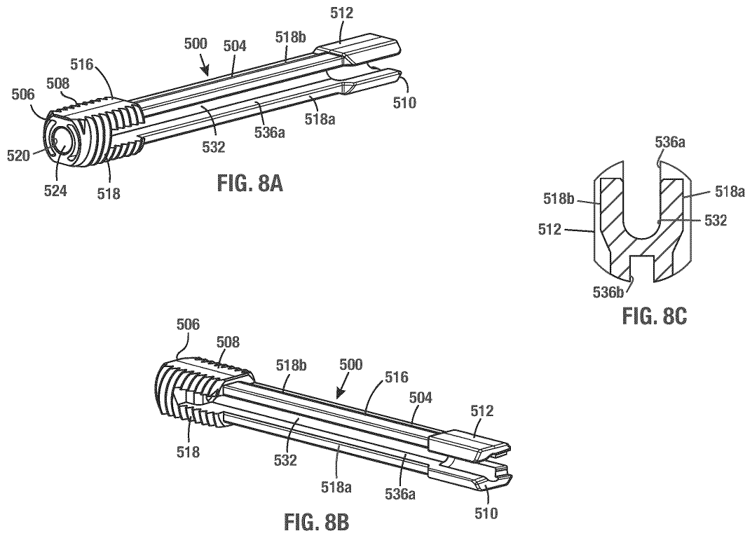

FIGS. 8A-8C illustrate an exemplary implementation of the carriage member 500. The carriage member 500 includes a carriage body 504 having a distal end 506 and a proximal end 510. The carriage body 504 has a head portion 508 and a stem portion 512 between the distal end 506 and the proximal end 510. The carriage body 504 can be formed (e.g., molded) as a single, unitary component. Preferably, the carriage body 504 has enough rigidity to support a portion of the shaft assembly received within the handle body 304 (shown in FIGS. 7B and 7C).

The head portion 508 of the carriage body 504 has an external surface 516. External threads 518 are formed on the portion of the external surface 516 at opposite sides of the head portion 508. The external threads 518 can engage a complementary internal thread in the drive member 320 (shown in FIGS. 7B and 7C) of the handle. The head portion 508 has an internal surface 520 that defines an internal bore 524 configured to receive a portion of the shaft assembly.

The stem portion 512 includes a central opening 532, which is longitudinally aligned with and connected to the internal bore 524 of the head portion 508, forming a passage extending along the entire length of the carriage body 504. Longitudinal slots 536a, 536b (or guide members) are formed on opposite sides of the stem portion 512. The longitudinal slot 536a may be connected to the central opening 532 (or to the passage formed by the bore 524 and central opening 532) as illustrated in FIG. 8C. The longitudinal slots 536a, 536b can receive complementary guide members 348a, 348b (shown in FIGS. 11A and 11B) within the elongated cavity of the handle body.

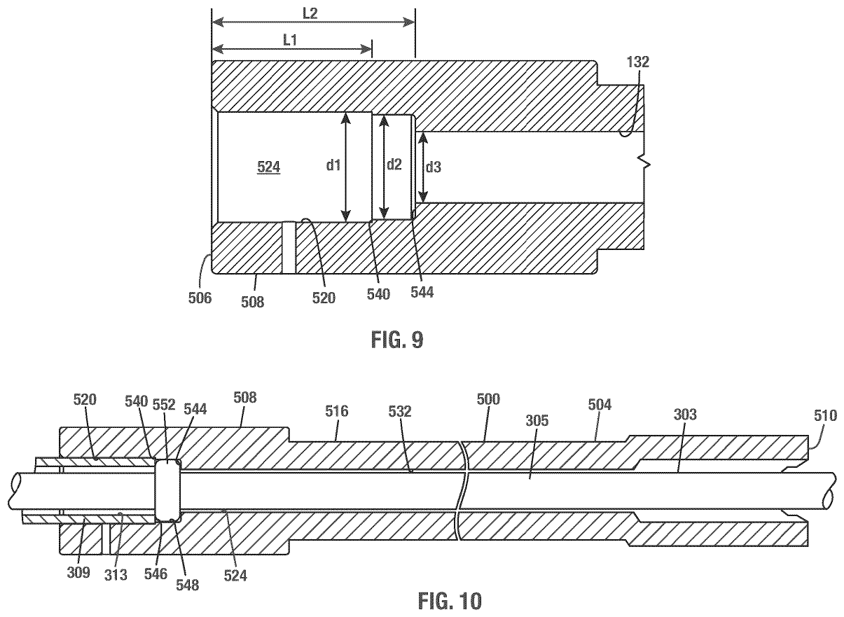

Referring to FIG. 9, a locating shoulder 540 is formed on the internal surface 520 of the head portion 508. The locating shoulder 540 defines a first stepdown transition in the internal bore 524. For example, the locating shoulder 540 steps down the diameter of the internal bore 524 from diameter d1 to diameter d2, where the diameter d1 is greater than the diameter d2. The locating shoulder 540 is offset from the distal end 506 of the carriage body 504 by a distance L1. The locating shoulder 540 has an annular face that is oriented towards the distal end 506 and may be referred to as “a distally facing annular shoulder” in some cases.

A gland shoulder 544 is formed on the internal surface 520 of the head portion 508. The gland shoulder 544 defines a second stepdown transition in the internal bore 524. For example, the gland shoulder 544 steps down the diameter of the internal bore 524 from diameter d2 to diameter d3, where the diameter d2 is greater than the diameter d3. The gland shoulder 544 is offset from the distal end 506 of the carriage body 504 by a distance L2 that is greater than the distance L1, which means that the gland shoulder 544 is located proximally to the locating shoulder 540. The gland shoulder 544 has an annular face that is oriented towards the distal end 506 and may be referred to as “a distally facing annular shoulder” in some cases.

FIG. 10 shows the shaft assembly 303 extending through the passage formed by the internal bore 524 and the central opening 532 such that the proximal end (or proximal face) of the outer shaft 309 is positioned within the internal bore 524. The proximal end of the outer shaft 309 forms a shoulder 546 that is in opposing relation to and distal relative to the gland shoulder 544. The outer shaft 309 can be secured to the head portion 508 of the carriage member 500 in this position (e.g., via fasteners, adhesive, and/or other means for coupling). An annular groove 548 (or gland) is defined within the internal bore 524 by the opposed shoulders 544, 546 and the portion of the internal surface 520 between the opposed shoulders 544, 546. The annular groove 548 can receive a seal member 552.

In some examples, the locating shoulder 540 can act as a stop surface for the proximal end of the outer shaft 309. In this case, the diameter d2 (shown in FIG. 9), which corresponds to the inner diameter of the locating shoulder 540, can be selected to be larger than an inner diameter of the outer shaft 309 at the proximal end of the outer shaft 309 such that when the proximal end of the outer shaft 309 abuts the locating shoulder 540, a portion of the proximal end of the outer shaft 309 forms the shoulder 546 at the first stepdown transition. As shown for example in FIG. 10, the shoulder 546 formed by the proximal end of the outer shaft 309 can be radially inward of the locating shoulder 540 at the first stepdown transition.

In other examples, the carriage body 504 can be formed without the locating shoulder 540, and the outer shaft 309 can be inserted into the internal bore 524 to a point at which the proximal face of the outer shaft 309 abuts the distal face of the seal member 522, which would at the same time form the distal end of the annular groove 548.

As illustrated by FIG. 10, the inner shaft 305 extending through the lumen of the outer shaft 309 passes through the portion of the internal bore 524 between the opposed gland shoulders 544, 546, which means that the annular groove 548 is disposed around a circumference of the inner shaft 305. Thus, the seal member 552 disposed in the annular groove 548 can form a seal between the inner shaft 305 and the internal surface 520 and at the proximal end of the outer shaft 309. The seal member 552 can cycle between dynamic sealing and static sealing. Dynamic sealing occurs when the seal member 552 slides along the inner shaft 305 as the carriage member 500 moves relative to the handle body 304 (shown in FIGS. 7B and 7C). In this manner, the seal member 552 can also be referred to as “a wiper seal.” The seal member 552 can be any suitable seal (e.g., an O-ring).

The gland shoulder 544 forms the proximal end of the annular groove 548 (or the proximal gland shoulder), and the proximal end (or proximal face) of the outer shaft 309 forms the distal end of the annular groove 548 (or the distal gland shoulder). In some cases, the locating shoulder 540 can form a stop for the outer shaft 309. Forming the shoulders of the carriage body as stepped shoulders can, among other things, allow the carriage body 504 (or carriage member 500) to be molded as a single piece. The molding process can include forming a mold cavity for the carriage body and a core pin to form the internal bore including the locating and gland shoulders 540, 544. The core pin is secured within the mold cavity, and molten thermoplastic material is injected into the mold cavity to form the molded body. The stepped shoulders can, for example, allow the core pin to be easily removed from the distal end of the molded part. As such, the disclosed configuration simplifies both manufacture and assembly of the handle as one exemplary advantage.

Returning to FIG. 7C, the carriage member 500 is axially movable within the cavity 316 and relative to the handle body 304 by rotation of the drive member 320. In the example illustrated by FIG. 11A, the drive member 320 has a barrel portion 320a extending into the cavity 316 from the distal end 312 of the handle body 304 and a knob portion 320b projecting from the distal end 312 of the handle body 304. The barrel portion 320a has a ring member 332 that extends into a recess 336 in the handle body 304. A distal face of the ring member 332 can abut a proximal face of the recess 336 to limit movement of the drive member 320 in the distal direction.

The drive member 320 includes an internal surface 328 that defines an internal bore 340. The internal surface 328 includes an internal thread 344, which is complementary to the external threads 518 (shown in FIGS. 8A and 8B) on the head portion of the carriage member 500. As shown, the carriage member 500 extends into the internal bore 340 such that the external threads 518 on the head portion of the carriage member 500 are engaged with the internal thread 344 in the drive member 320.

Rotation of the knob portion 320b causes rotation of the drive member 320 relative to the handle body 304, which causes the carriage member 500 to move along the internal bore 340 of the drive member 320. The threads 344, 518 translate the rotary motion of the drive member 320 to a linear motion of the carriage member 500. However, other mechanisms besides a lead screw mechanism can be used to translate the carriage member 500 axially relative to the handle body 304.

Referring to FIGS. 11A and 11B, the handle body 304 can include flattened projections 348a, 348b (or guide members) extending into the cavity 316. The flattened projections 348a are received in the longitudinal slot 536a of the carriage member 500. The flattened projection 348b is received in the longitudinal slot 536b. The longitudinal slots 536a, 536b move along respective flattened projections 348a, 348b as the carriage member 500 moves axially within the cavity 316 and relative to the handle body 304. The flattened projections 348a, 348b are longitudinally aligned with the handle body 304 and cooperate with the longitudinal slots 536a, 536b to prevent rotation of the carriage member 500 when the drive member 320 is rotated.

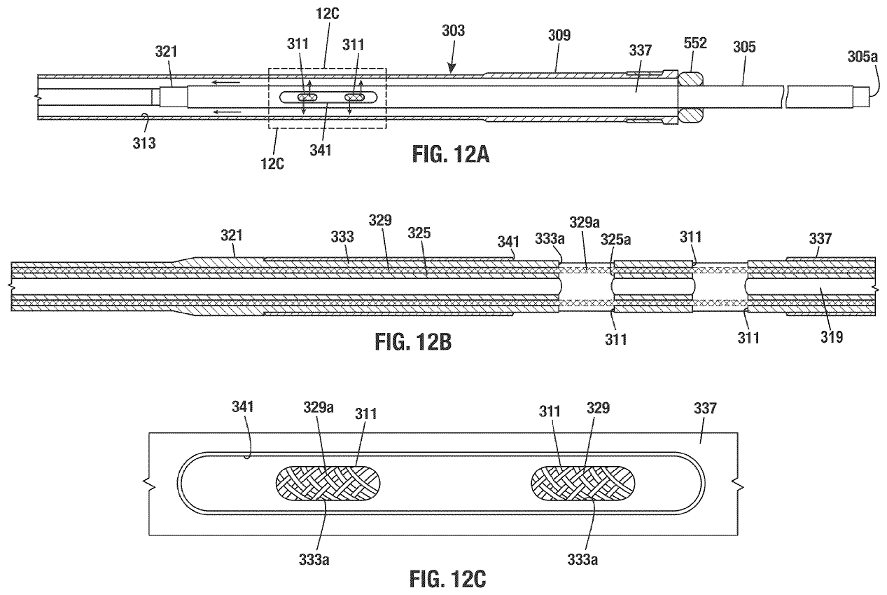

FIG. 12A shows a proximal portion of the shaft assembly 303 (i.e., the portion of the shaft assembly 303 immediately coupled to the handle). The proximal portion of the shaft assembly 303 includes a proximal portion of the outer shaft 309 and a proximal portion of the inner shaft 305 extending through the lumen 313 of the outer shaft 309. As previously described with respect to FIG. 11A, the proximal end of the outer shaft 309 is received within the carriage member 500, and the inner shaft 305 extends through the outer shaft 309 and through the carriage member. As shown in FIG. 12A, the proximal end portion of the inner shaft 305 includes the proximal end 305a that can be fluidly connected to the injection port 324 (shown in FIGS. 7A-7C and 11A) and the fluid ports 311 that allow fluid injected into the lumen 319 of the inner shaft 305 at the injection port to exit the lumen 319 of the inner shaft 305 and enter the lumen 313 of the outer shaft 309.

In one implementation, the inner shaft 305 includes a reinforced tube 321. In the example illustrated by FIG. 12B, the reinforced tube 321 can include an inner layer 325, a reinforcement layer 329 disposed over the inner layer 325, and an outer layer 333 disposed over the reinforcement layer 329. The inner layer 325, the reinforcement layer 329, and the outer layer 333 can be in the form of tubes extending substantially along the length of the inner shaft 305.

The reinforced tube 321 can be configured as a flexible tube to facilitate movement of the tube through the vasculature of a patient. The reinforcement layer 329 can be, for example, a braided tube, which can be made from metal wire (such as stainless-steel wire or Nitinol wire) or from synthetic fibers or combinations thereof. The inner layer 325 and the outer layer 333 can be tubes made of a polymer material. Examples of suitable polymer materials include, but are not limited to, PEBAX® elastomers, nylons, and polyurethane. The inner layer 325 and outer layer 333 can be made of the same material or of different materials. In some cases, the reinforced tube 321 can be made by extrusion.

The inner shaft 305 can include one or more fluid ports. The fluid ports are formed in the wall of the reinforced tube and can allow a flushing fluid to flow from the inner lumen 319 of the inner shaft 305 and into the lumen 313 of the outer shaft 309. In this manner, the fluid ports 311 enable flushing of the inner shaft 305 and the outer shaft 309 from a single injection port rather than requiring the shafts to be separately flushed. Referring to FIGS. 12B and 12C, each fluid port 311 includes a first opening 325a in the inner layer 325, a second opening 333a in the outer layer 333 that is radially aligned with the first opening, and the pores (or openings) in the portion 329a of the reinforcement layer 329 between the two openings 325a, 333a. The openings 325a, 333a can have any suitable shape (e.g., oval as shown in FIGS. 12A and 12C, circular, square, or rectangular shape).

Any number of fluid ports 311 can be formed in the reinforced tube 321. For example, the illustrated reinforced tube 321 comprises four ports 311 (shown in FIG. 12B). When there are multiple fluid ports 311, various arrangements of the fluid ports 311 on the reinforced tube 321 are possible. For example, FIGS. 12A-12C show two of the fluid ports 311 spaced apart axially and aligned circumferentially along the reinforced tube 321. As depicted in FIG. 12B, the reinforced tube 321 also comprises two additional fluid ports 311 that are axially aligned and circumferentially spaced apart (e.g., by 180 degrees) from the fluid ports depicted in FIG. 12C. In another example, the fluid ports 311 can be spaced apart and/or staggered around the reinforced tube 321. For example, the fluid ports 311 can be spaced apart and staggered around the reinforced tube 321 to form a spiral pattern. In another example, the fluid ports can form an alternating-type pattern such that a first side of the tube comprises a plurality of ports (e.g., a first proximal port and a first distal port), and a second side of the tube (e.g., located 180 degrees from the first side) comprises a plurality of ports (e.g., a second proximal port and a second distal port), and the ports are arranged axially in the following manner moving proximal to distal: first proximal port, second proximal port, first distal port, second distal port.

The inner shaft 305 can, in some instances, include a cover tube 337 extending over a proximal portion of the reinforced tube 321. The cover tube 337 includes one or more windows 341 positioned to expose the fluid ports 311. The cover tube 337 is the part of the inner shaft 305 that contacts the seal member 552 (shown in FIG. 11A) when the inner shaft 305 extends through the carriage member 500 (shown in FIG. 11A). The cover tube 337 is preferably a rigid member that can support sliding of the seal member. The cover tube 337 preferably has a surface finish to provide a proper sealing surface to the seal member 552. The cover tube 337 can be made of metal or plastic. For example, the cover tube 337 can be made from stainless steel. The cover tube 337 can be secured to the reinforced tube 321 by any suitable method, such as by crimping, adhesive, etc.

The inner shaft 305 can be formed using any suitable method. One preferred method for forming the inner shaft 305 includes initially providing the reinforced tube 321 without the openings that form the fluid ports. The cover tube 337 with the one or more windows 341 formed therein is disposed over the reinforced tube 321 and secured to the outer layer 333 of the reinforced tube 321. The fluid ports 311 are then formed in the area of the reinforced tube 321 exposed through the window(s) 341 of the cover tube 337.

In one example, the openings that form each fluid port 311 are formed in the outer layer 333 and the inner layer 325 of the reinforced tube 321 by laser ablation. Advantageously, the laser beam used in the laser ablation can be configured to remove material from only portions of the outer layer 333 and the inner layer 325 of the reinforced tube 321, leaving the reinforcement layer 329 intact to maintain the tensile strength of the reinforced tube 321. In addition, the laser ablation process removes material by vaporizing the material, thereby reducing or eliminating potential particulate contamination of the delivery apparatus. Any film that settles on the surfaces of the tube can be flushed out.

Referring to FIGS. 11A and 12A, fluid (e.g., saline) can be injected into the lumen 319 of the inner shaft 305 through the injection port 324 for the purpose of flushing the inner shaft. The fluid will move through the lumen 319 of the inner shaft 305. A portion of the fluid moving through the lumen 319 of the inner shaft 305 will exit through the fluid ports 311 and enter the lumen 313 of the outer shaft 309, allowing flushing of the outer shaft. Thus, both the inner shaft 305 and the outer shaft 309 can be flushed using a single injection port. The seal member 552 forms a seal at the proximal end of the outer shaft 309 and prevents leakage of the fluid from the proximal end of the outer shaft. Later, during use of the delivery apparatus, the seal member 552 will also prevent leakage of blood from the proximal end of the outer shaft, thereby maintaining hemostasis.

Returning to FIGS. 6A-6F, the docking station 136 can be configured as a self-expanding docking station where the docking station 136 and the connector tabs 132 are naturally biased toward an expanded configuration. While the docking station 136 is attached to the delivery system, the docking station 136 is compressed to a smaller configuration (shown in FIG. 6B) for insertion and tracking through the vasculature. The compressed configuration of the docking station is held in place axially by the frame connector 400 (which is fixed relative to the inner shaft 305) and held in place radially by the outer shaft 309. The docking station 136 is therefore prevented from premature deployment by the frame connector 400 and the outer shaft 309. Once the docking station 136 is at the implantation location within the anatomy, the outer shaft 309 can be retracted to expose and deploy the docking station 136.

As the outer shaft 309 is retracted to expose the docking station 136, the distal portion of the docking station 136 expands (as shown, for example, in FIGS. 6C and 6D). In some cases, prior to completing retraction of the outer shaft 309, it may be desirable to reposition and/or retrieve the docking station 136. In this case, the outer shaft 309 may be extended again to recapture and recompress the docking station 136 in order to allow the docking station 136 to be repositioned and/or retrieved. However, the bias toward an expanded configuration can create an axial tension between the docking station and the frame connector. The axial tension can concentrate at the flanges of the connector tabs of the docking station as the outer shaft is extended distally over the docking station for recapture. Due to the relatively high forces during recapture and/or retrieval, the connector tabs of the docking station tend to move radially outwardly attempting to disengage from the frame connector 400. This can increase the force required to recapture the docking station. In extreme instances, the connector tabs can disengage from the connector, which can inhibit recompression and/or retrieval of the docking station.

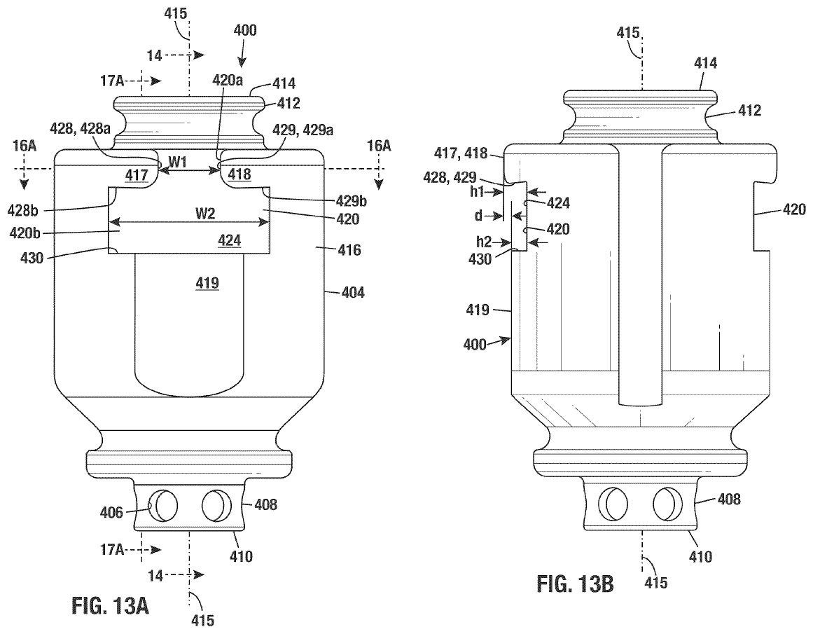

FIGS. 13A-17B illustrate an exemplary implementation of the frame connector 400 that can help retain the connector tabs in the radially-compressed configuration during recompression/retrieval of the docking station. Referring to FIGS. 13A and 13B, the frame connector 400 includes a connector body 404, a flange 408 attached to one end of the connector body 404, and a flange 412 attached to another end of the connector body 404. The flange 408 provides a proximal end 410 of the connector, and the flange 412 provides a distal end 414 of the connector. The frame connector 400 has a longitudinal axis 415 (or central axis) extending from the proximal end 410 to the distal end 414. The longitudinal axis 415 defines the axial direction of the connector.

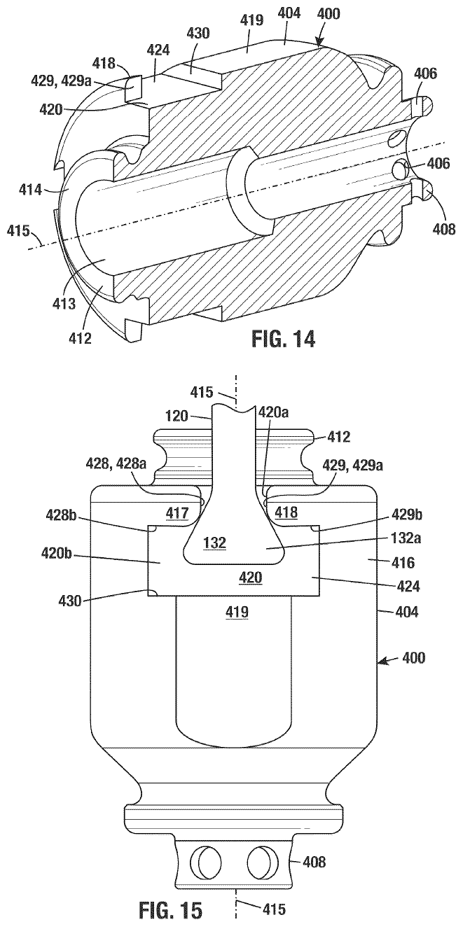

As shown in FIG. 14, the frame connector 400 has an internal bore 413 extending through the flanges 408, 412 and connector body 404 and along the longitudinal axis (415 in FIG. 13B). The internal bore 413 can receive a proximal portion of the inner shaft of the shaft assembly of the delivery apparatus. The flange 408 can include radial holes 406 that connect to the internal bore 413. As will be described later, the radial holes 406 can play a role when the frame connector 400 is fixed to the inner shaft of the shaft assembly (e.g., by an over-molding process).

Returning to FIGS. 13A and 13B, the connector body 404 includes an exterior with an exterior surface 416 and one or more recesses 420. Each of the recesses 420 can receive one of the connector tabs of the docking station. In the implementation illustrated by FIGS. 13A-17B, two recesses 420 are formed in diametrically opposed positions on the exterior of the connector body 404. In general, when a plurality of recesses 420 are formed on the exterior of the connector body 404, the recesses 420 can be formed in angularly (which may also be referred to as “circumferentially”) spaced apart locations along the exterior of the connector body 404 (i.e., distributed along a circumference of the connector body 404).

Referring still to FIGS. 13A and 13B, each recess 420 can be a recessed slot having a first slot portion 420a and a second slot portion 420b arranged to form a “T” shape. As shown, the first slot portion 420a is generally aligned with the longitudinal axis 415 of the connector and is generally perpendicular to the second slot portion 420b. The first slot portion 420a has a first width W1, and the second slot portion 420b has a second width W2. The second width W2 is greater than the first width W1, which means that the recess 420 transitions from a larger width slot portion 420b to a smaller width slot portion 420a. As shown in FIG. 15, the recess 420 is open at the exterior surface 416 so that a connector tab 132 having a flared portion 132a can be positioned in the recess from the exterior surface 416.

Referring to FIGS. 13A and 16A, each recess 420 has a recess floor 424, opposite side walls 428, 429, and an end wall 430. The side walls 428, 429 project from opposite sides of the recess floor 424. The side wall 428 is connected to a portion 417 of the exterior surface 416. The side wall 429 is connected to a portion 418 of the exterior surface 416. The end wall 430 projects from an end of the recess floor 424 and is connected to a portion 419 of the exterior surface 416. The recess floor 424 is on a different plane compared to the surface portions 417, 418, 419. In particular, the recess floor 424 is recessed (or radially inward) relative to the surface portions 417, 418, 419, as shown more clearly in FIG. 16A.

In one example, the surface portions 417, 418 are on the same plane but on a different plane compared to the surface portion 419. For example, as shown in FIG. 13B, each of the surface portions 417, 4-18 can be radially outward of the surface portion 419 by an offset distance d. Stated differently, the height h1 of the side walls 428, 429 relative to the recess floor 424 can be greater than the height h2 of the end wall 430 relative to the recess floor 424. Since the connector tab that is received in the recess 420 will contact the side walls 428, 429, the height of the side walls 428, 429 can be selected to provide sufficient engagement surfaces for the connector tab.

A first portion 428a of the side wall 428 and a first portion 429a of the side wall 429 form opposite sides of the first slot portion 420a (in FIG. 13A) of the recess 420. The end wall 430 is longitudinally displaced from the first and second walls 428, 429 by a distance that determines the height of the second slot portion 420b (in FIG. 13A) of the recess 420. A second portion 428b of the side wall 428 and a second portion 429b of the side wall 429 are in opposing relation to the end wall 430. The end wall 430 and the second portions 428b, 429b of the side walls 428, 429 form opposite ends of the second slot portion 420b of the recess 420.

FIG. 15 shows a connector tab 132 of a docking station positioned within a recess 420 of the frame connector 400 prior to deployment of the docking station at an implantation location. The connector tab 132 can be formed at an apex of a strut 120 of the frame of the docking station as previously described. In the example illustrated by FIG. 15, the connector tab 132 has a flared portion 132a that sits in the second slot portion 420b and engages the side walls 428, 429. The flared portion 132a engages the side walls 428, 429 because the flared portion 132a is wider than the first slot portion 420a. When the flared portion 132a engages the side walls 428, 429 as shown, the connector tab 132 is prevented from being pulled axially through the first slot portion 420a.

To help retain the connector tab 132 in the radially-compressed configuration and thus its connection with the frame connector 400 when axial tension is created between the docking station and the frame connector, the second portions 428b, 429b of the side walls 428, 429 are formed as undercut walls, which means that there is a space or recess underneath each of the second portions 428b, 429b (or a space or recess between each of the second portions 428b, 429b and the recess floor 424). As illustrated in FIGS. 17A and 17B, the second portions 428b, 429b, which are formed as undercut walls, are inclined relative to the recess floor 424 (i.e., the second portions 428b, 429b are not perpendicular to the recess floor 424). The angle α between the second portion 428b and the recess floor 424 is less than 90 degrees, and the angle θ between the second portion 429b and the recess floor 424 is less than 90 degrees. In some examples, each of the angles α and θ can be in a range of 45-89.9 degrees. In other examples, each of the angles α and θ can be in a range of 75-89.9 degrees. In one preferred example, each of the angles α and θ can be in a range of 81-86 degrees. The angles α and θ can be the same or can be different.

When the frame connector 400 as illustrated by FIGS. 17A and 17B is used to axially restrain the docking station 136, the tensile force created by the bias of the docking station to the expanded configuration pulls the flared portion (132a in FIG. 15) of the connector tab axially against the second portions 428b, 429b. The undercut in the second portions 428b, 429b translates a portion of the tensile force into a radial force that pushes the connector tab radially inwardly toward the central axis of the frame connector 400, thereby improving retention characteristics of the docking station prior to deployment of the docking station. It has been found that each of the angles α, θ between the second portions 428b, 429b and the recess floor 424 in a range of 81-86 degrees (in certain instances) improves securement of the docking station to the delivery system when the outer shaft is extended during recapture of the docking station.

Returning to FIGS. 13A and 16A, the first portions 428a, 429a can be formed as undercut walls, which means that there is a space or recess underneath each of the first portions 428a, 429a (or a space or recess between each of the first portions 428a, 429a and the recess floor 424). As illustrated in FIG. 16B, the first portions 428a, 428b as undercut walls are inclined relative to the recess floor 424 (i.e., the first portions 428a, 429b are not perpendicular to the recess floor 424). The angle β between the first portion 428a and the recess floor 424 is less than 90 degrees, and the angle φ between the first portion 429a and the recess floor 424 is less than 90 degrees. In some examples, each of the angles β and φ can be in a range of 45-89.9 degrees. In other examples, each of the angles β and φ can be in a range of 75-89.9 degrees. In one example, each of the angles β and φ can be in a range of 81-86 degrees. The angles β and φ can be the same or can be different. In some examples, the angles β and/or φ can be the same as the angles α and/or θ. In other examples, the angles β and/or φ can be different than the angles α and/or θ.

Returning to FIG. 13A, each of the side walls 428, 429 includes a corner where the first slot portion 420a is connected to the second slot portion 420b. These corners can be rounded and can have undercuts such that an undercut extends underneath the entire length of each of the side walls 428, 429. The edges where the side walls 428, 429 meet the exterior surface portions 417, 418 can be similarly rounded.

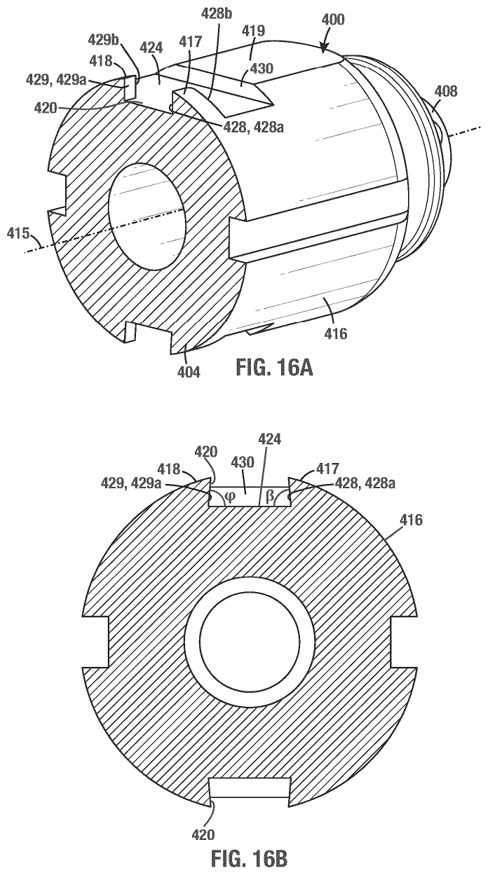

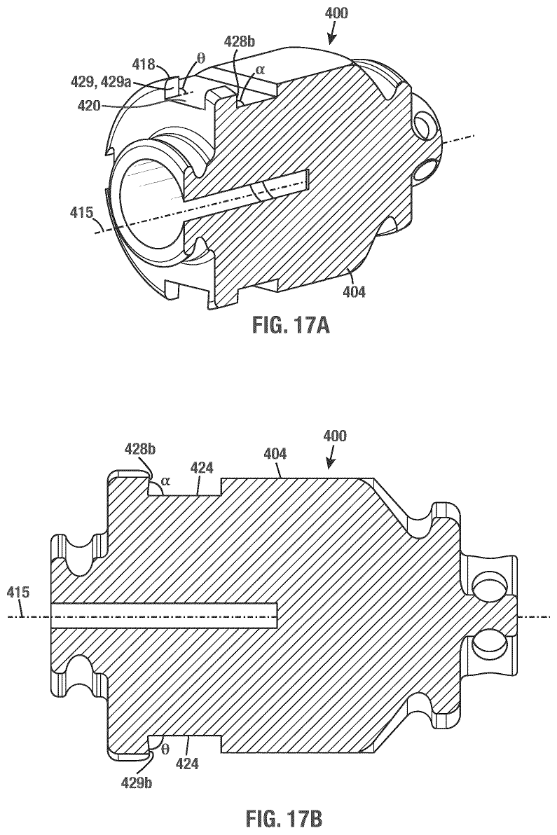

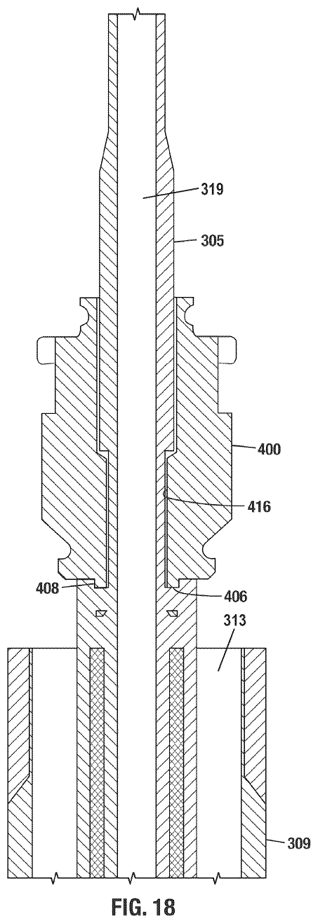

Referring to FIG. 18, one preferred method of coupling the frame connector 400 to a distal portion of the inner shaft 305 (shown in FIG. 5A) is by an over-molding process. During the over-molding process, the radial holes 406 in the flange 408 can receive flow of injected material. The material in the radial holes 406, when solidified, can anchor the frame connector 400 to the inner shaft 305. FIG. 18 shows the inner shaft 305 extending through the lumen 313 of the outer shaft 309. The frame connector 400 is sized relative to the outer shaft 309 such that the outer shaft 309 can be extended over the frame connector 400 and over a docking station disposed around a portion of the inner shaft 305 distal to the frame connector 400.

FIGS. 19 and 20 illustrate a portion of the delivery apparatus 300 including the docking station 136 in a compressed configuration. The outer shaft 309 is extended to encapsulate the docking station 136. Each of the connector tabs 132 of the docking station 136 is disposed in a respective recess 420 of the frame connector 400 and engaged with the side walls of the recess 420. The docking station 136 is held in place axially by the frame connector 400 and radially by the outer shaft 309. It should be understood that only a portion of the delivery apparatus is shown in FIGS. 19 and 20. The remaining portions of the delivery apparatus (e.g., the portion that extends to the nosecone, the portion that is coupled to the handle, the nosecone, and the handle) are visible in FIG. 5A.

A delivery assembly that is configured as shown in FIGS. 19 and 20 can be inserted into a patient's body and advanced through the patient's vasculature to an implantation location. At the implantation location, the outer shaft 309 can be retracted to expose the docking station 136 and deploy the docking station (as illustrated in FIGS. 6C-6F). During recapture of the docking station 136, the inner shaft 305 can be under high tensile loads while the outer shaft 309 is extended to cover docking station 136. The undercut in the side walls of the recess 420 can translate the tensile force acting on the respective connector tab 132 to a radial force that pushes the connector tab 132 inwardly toward the central axis of the frame connector 400, as illustrated in FIG. 21, thereby retaining the connection between the delivery apparatus and the docking station.

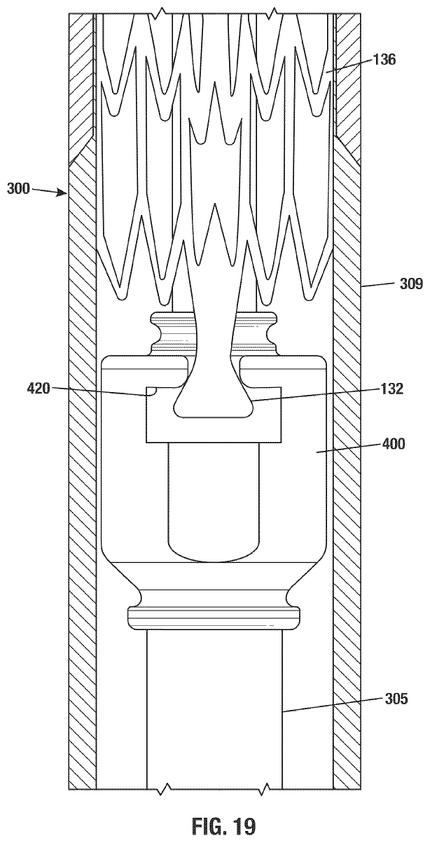

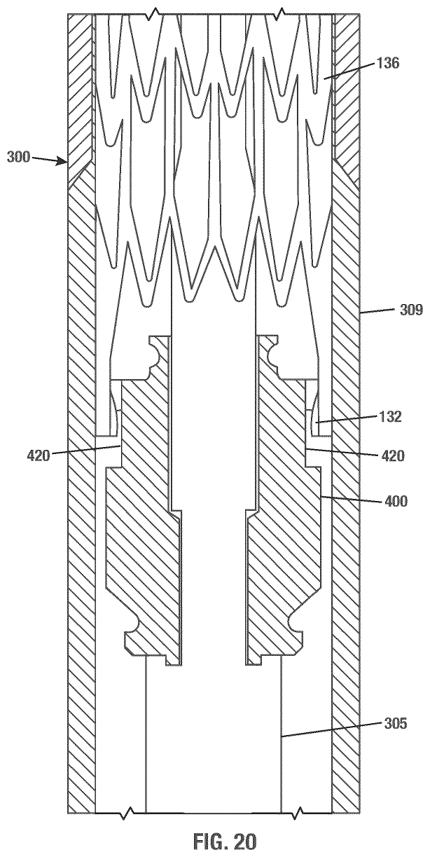

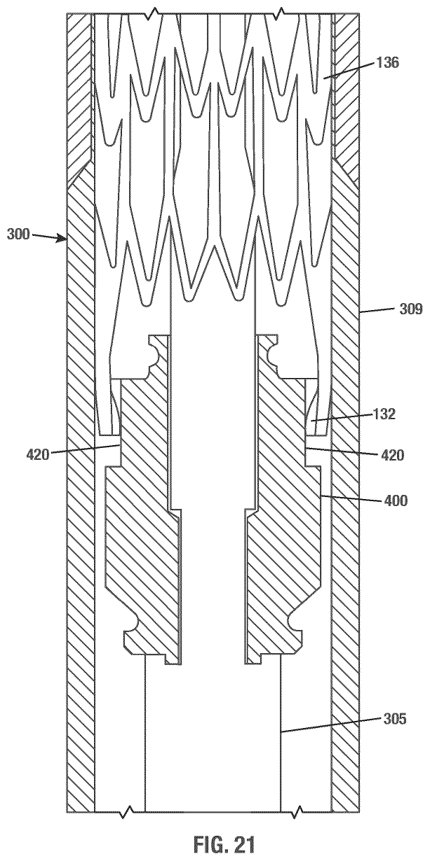

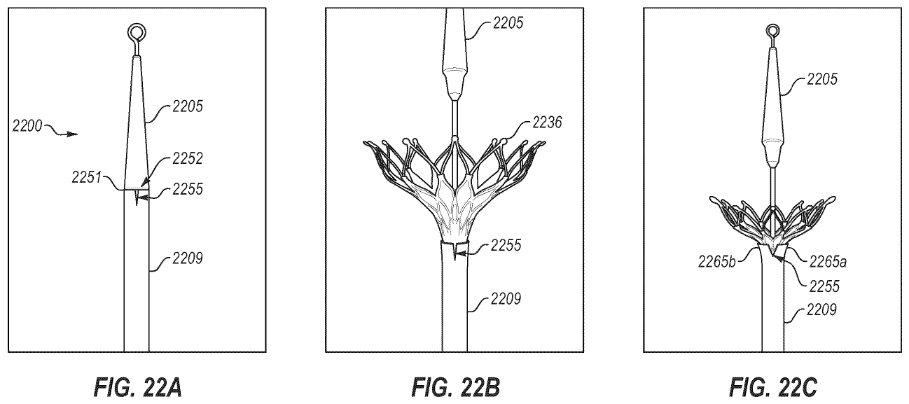

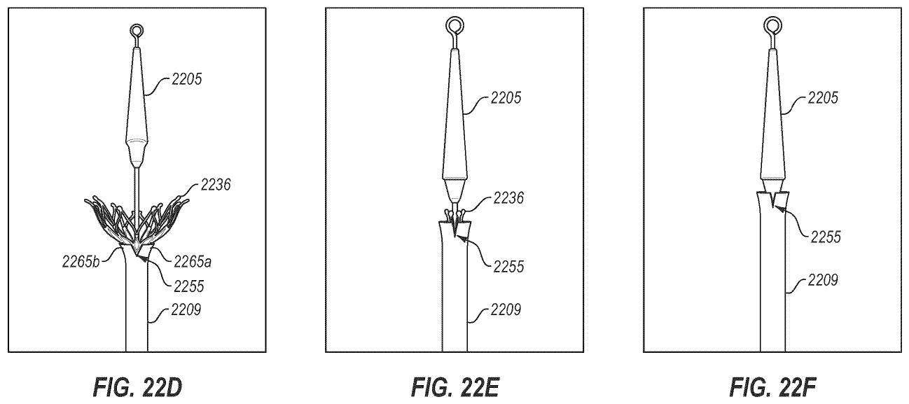

FIGS. 22A-22F illustrate operation of a delivery apparatus 2200 with an outer shaft 2209 that includes one or more flaring openings 2255 to facilitate retraction of an implant 2236. The delivery apparatus 2200 may include an inner shaft 2205 and an outer shaft 2209. The delivery apparatus 2200 may additionally include the implant 2236. The implant 2236 may be loaded onto the inner shaft 2205 and deployed within a patient to an implantation location, and the outer shaft 2209 may be retracted to expose the implant 2236.

The delivery apparatus 2200 may be similar or comparable to other delivery apparatuses of the present disclosure, such as the delivery apparatus 300. The inner shaft 2205 may be similar or comparable to other inner shafts of the present disclosure, such as the inner shaft 305. The outer shaft 2209 may be similar or comparable to other outer shafts of the present disclosure, such as the outer shaft 309. The implant 2236 may be similar or comparable to other implants of the present disclosure, such as the docking station 136. In some embodiments, the implant 2236 may be an expandable device that may expand upon retraction of the outer shaft 2209 and the corresponding exposure of the implant 2236.

The outer shaft 2209 as illustrated in FIGS. 22A-22F includes one or more flaring openings 2255. The flaring openings 2255 may facilitate an expansion of the outer circumference 2252 of the outer shaft 2209 at a distal end 2251 of the outer shaft 2209 by having one or more portions of the outer shaft 2209 at the distal end 2251 flaring radially outwards.

As illustrated in FIG. 22A, the implant 2236 may be fully enclosed within the outer shaft 2209. For example, the state of the delivery apparatus 2200 may be utilized as the delivery apparatus 2200 traverses the vasculature of the patient to arrive at the implantation location.

As illustrated in FIG. 22B, when the outer shaft 2209 is retracted, the implant 2236 may begin to expand into its deployed state. For example, as illustrated in FIG. 22B, the implant 2236 may be approximately 60% deployed.

In some embodiments, after an initial stage of deployment (e.g., at the stage illustrated in FIG. 22B), an individual operating the delivery apparatus 2200 may desire to retract or re-capture the implant 2236 back into the outer shaft 2209. For example, the individual may desire to reposition the implant 2236 and/or the delivery apparatus 2200 to a better or more precise location. As another example, the individual may desire to retrieve the implant 2236 if the implant 2236 is not deploying or expanding as expected.

As illustrated in FIG. 22C, as the outer shaft 2209 is extended back over the implant 2236, the flaring openings 2255 permit the distal end 2251 of the outer shaft 2209 to flare in a radially outward direction to more readily accommodate the implant 2236 being compressed back into its loaded state for traversing the vasculature of the patient. In some embodiments, multiple of the flaring openings 2255 may be included, thereby creating phalanges 2265 (such as the phalanges 2265a-b) at the distal end 2251 of the outer shaft 2209. In these and other embodiments, each of the phalanges 2265 may individually flare outwards.

FIG. 22C illustrates a state of the delivery apparatus when the implant 2236 is approximately 70% re-captured within the outer shaft 2209.

As illustrated in FIG. 22D, the implant 2236 may continue to be further re-captured within the outer shaft 2209. FIG. 22D illustrates a state of the delivery apparatus 2200 when the implant 2236 is approximately 80% re-captured within the outer shaft 2209.

FIG. 22E illustrates a state of the delivery apparatus 2200 when the implant 2236 is approximately 95% re-captured within the outer shaft 2209.

FIG. 22F illustrates a state of the delivery apparatus 2200 when the implant 2236 is approximately 100% or fully re-captured within the outer shaft 2209.

As illustrated in FIGS. 22A-22F, there are a number of transition points of the position of the phalanges 2265. For example, a first transition point may occur where the phalanges 2265 and corresponding outer circumference of the distal end 2251 of the outer shaft 2209 first begin to flare outwards, and a second transition point may occur where the phalanges 2265 and corresponding outer circumference of the distal end 2251 of the outer shaft 2209 stop the outward flaring, and begin to retract back inward in the radial direction towards the original shape and position of the phalanges 2265 at the distal end 2251 of the outer shaft 2209.

In some embodiments the first transition point may occur while the implant 2236 is being deployed, such as when it is 10% deployed, 20% deployed, 30% deployed, 40% deployed, 50% deployed, 60% deployed, 70% deployed, 80% deployed, or 90% deployed. In some embodiments, the first transition point may occur when the implant 2236 is starting to be re-captured. In these and other embodiments, the reversal of direction of moving the outer shaft 2209 relative to the inner shaft 2205 to begin the re-capturing of the implant 2236 may cause the first transition point where the phalanges 2265 begin to flare outwards.

In some embodiments, the second transition point may occur when the implant 2236 is sufficiently re-captured such that the phalanges 2265 begin to return to their original position as the implant 2236 is no longer pressing against the inner surface of the outer shaft 2209 to cause the outward flaring. Or stated another way, the displacing force of the interference of the implant 2236 with the phalanges 2265 may be lower than the retractive force of the material of the outer shaft 2209 at or after the second transition point such that the phalanges 2265 of the outer shaft 2209 begin to return to their original position before flaring radially outward. In some embodiments, the second transition point may occur when the implant 2236 is 20% re-captured, 30% re-captured, 40% re-captured, 50% re-captured, 60% re-captured, 70% re-captured, 80% re-captured, 90% re-captured, or 100% re-captured.

In some embodiments, the angle between the phalanges 2265 and the implant 2236 may shift and vary throughout the deployment and/or re-capture of the implant 2236. The change in angle may vary based on a shape or profile of the implant 2236 and a stage and direction of deployment and/or re-capture.

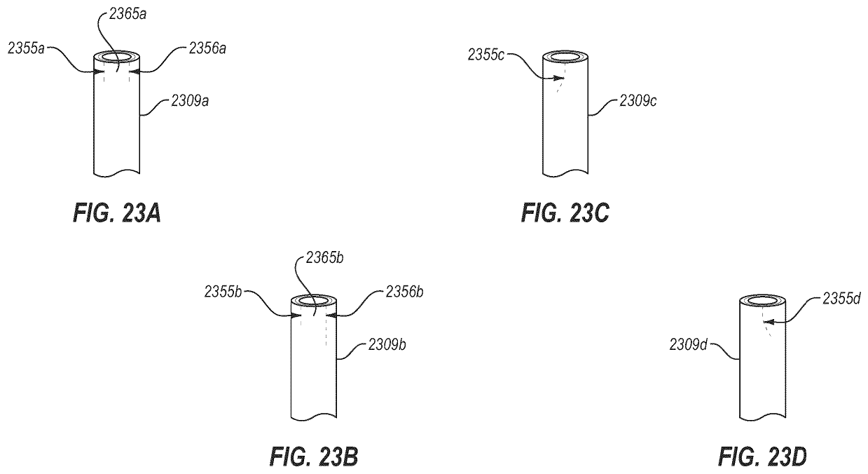

FIGS. 23A-23D illustrate perspective views of various embodiments of outer shafts 2309 (such as the outer shafts 2309a-2309d corresponding to FIGS. 23A-23D, respectively) that include one or more flaring openings 2355 and/or 2356 (such as slits 2355a and 2356a in FIG. 23A, slits 2355b and 2356b in FIG. 23B, slit 2355c in FIG. 23C, and/or slit 2355d in FIG. 23D). The flaring openings 2355 and/or 2356 may form one or more phalanges 2365 (such as the phalanges 2365a-2365b of FIGS. 23A-23B, respectively).

The outer shafts 2309 may be similar or comparable to other outer shafts of the present disclosure, such as the outer shafts 2209 or 309. Additionally or alternatively, the flaring openings 2355 and/or 2356 may be similar or comparable to other flaring openings, such as the flaring opening 2255. Additionally or alternatively, the phalanges 2365 may be similar or comparable to other phalanges, such as the phalanges 2265.

As illustrated in FIG. 23A, in some embodiments, the flaring openings 2355 may include two slits 2355a and 2356a extending in a generally longitudinal direction along a length of the outer shaft 2309a. The slits 2355a and 2356a may extend in a radial direction through the entire thickness of the outer shaft 2309a. In some embodiments, the two slits 2355a and 2356a may originate at the distal end of the outer shaft 2309a and extend in a generally parallel direction. The two slits 2355a and 2356a may be approximately the same length. In these and other embodiments, the two slits 2355a and 2356a together may form the phalange 2365a. The two slits 2355a and 2356a may permit the phalange 2365a to flare radially outward. When flaring outward, an initial point of deviance of the phalange 2365a away from a profile of the rest of the outer shaft 2309a may begin at approximately the end of the two slits 2355a and 2356a opposite the distal end of the outer shaft 2309a.

As illustrated in FIG. 23B, in some embodiments, the flaring openings 2355 may include two slits 2355b and 2356b extending in a generally longitudinal direction along a length of the outer shaft 2309b in a similar or comparable manner to that described with reference to FIG. 23A. The slit 2355b may be shorter in longitudinal length than the slit 2356b. By providing different lengths of slits, the phalange 2365b may flare outwards in a different manner than in the embodiment illustrated in FIG. 23A. For example, the phalange 2365b may flare radially outwards but at an angle offset from the longitudinal direction of the outer shaft 2309b. By opening at an angle, one transverse direction may have a wider opening at the distal end of the outer shaft 2309 than another transverse direction. For example, the outer shaft 2309b may include four slits in a symmetrical pattern of a pair of the two slits 2355b and 2356b. In such a configuration, the distal end of the outer shaft 2309b may open more widely in the transverse direction going through the slit 2356b than in the transverse direction going through the slit 2355b.

In some embodiments, the flaring openings 2355 and/or 2356 may be of any length. For example, the flaring openings 2355 and/or 2356 may be described in length relative to a diameter of the outer shaft 2309. In some embodiments the flaring openings 2355 and/or 2356 may be between approximately 0.5× and 2× the diameter of the outer shaft 2309. In some embodiments, the flaring openings 2355 and/or 2356 may be between approximately 0.5× and 0.75×, between approximately 0.75× and 1.0×, between approximately 1.0× and 1.25×, between approximately 1.25× and 1.5×, between approximately 1.5× and 1.75×, between approximately 1.75× and 2.0×, or any ranges therebetween and/or bounded by any numerical range identified herein.

As illustrated in FIGS. 23C and 23D, in some embodiments, the flaring openings 2355 may have a curved profile, such as the slits 2355c and/or 2355d. For example, as the slits 2355c and/or 2355d progress along a length of the outer shaft 2309c/2309d, the slits 2355c and/or 2355d may travel in both a longitudinal direction and a circumferential direction relative to the outer shaft 2309c/2309d. For example, the slit 2355c travels in a first circumferential direction, and the slit 2355d travels in a second, opposite circumferential direction. Additionally, while two example curve profiles are illustrated, it will be appreciated that other curve profiles are contemplated, such as a straight profile for a first span and then curving, curving for a first span and then proceeding in a straight profile, a profile with a steeper curve, a profile with a more shallow curve, a profile with a first curve for a first span and then a steeper curve in a second span, a profile with a first curve for a first span and then a more shallow curve in a second span, among others.

While various examples of different variations on combinations of slits and lengths, are illustrated, it will be appreciated that the present disclosure includes other combinations and variations. For example, the present disclosure specifically contemplates combinations of curved profile and straight profile slits, slits of different lengths (whether curved profile or straight profile), and any number of slits.

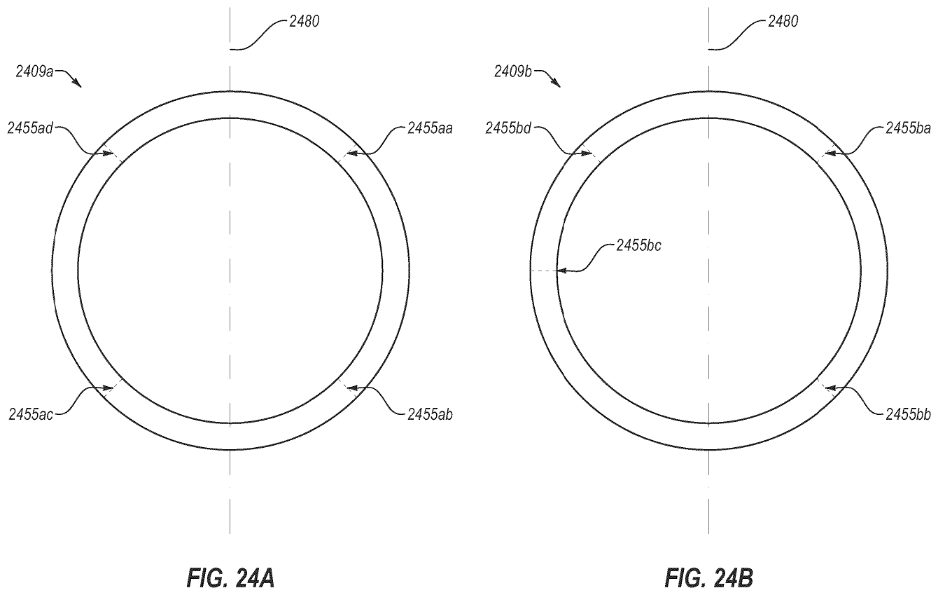

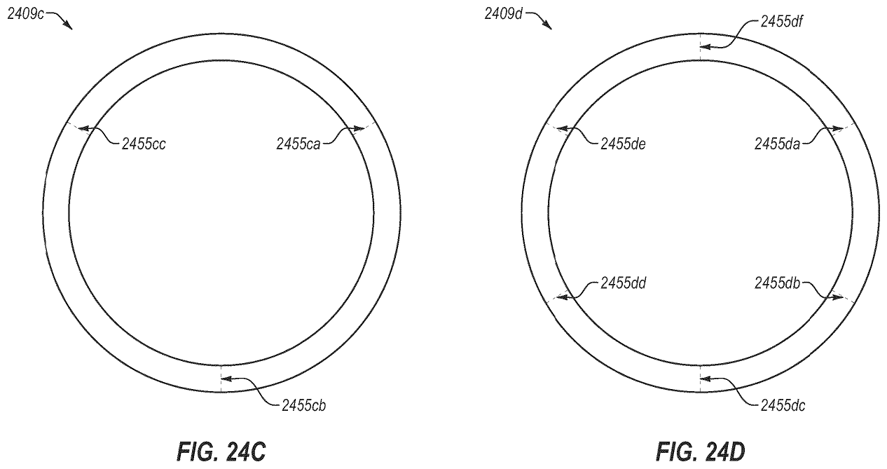

FIGS. 24A-24D illustrate longitudinal views of various configurations of embodiments of an outer shaft 2409 with multiple flaring openings 2455. For example, the outer shaft 2409a is illustrated as including the slits 2455aa, 2455ab, 2455ac, and 2455ad. As another example, the outer shaft 2409b is illustrated as including the slits 2455ba, 2455bb, 2455bc, and 2455bd. As a further example, the outer shaft 2409c is illustrated as including the slits 2455ca, 2455cb, and 2455cc. As an additional example, the outer shaft 2409d is illustrated as including the slits 2455da, 2455db, 2455dc, 2455dd, 2455de, and 2455df.

The outer shafts 2409 may be similar or comparable to other outer shafts of the present disclosure, such as the outer shafts 2209, 2309, or 309. Additionally or alternatively, the flaring openings 2455 may be similar or comparable to other flaring openings, such as the flaring opening 2255, 2355, and/or 2356. For example, while illustrated from the distal end of the outer shaft 2409, it will be appreciated that the flaring openings 2455 may include a straight profile or a curved profile and may travel for any length as disclosed herein.

As illustrated in FIG. 24A, the slits 2455aa-2455ad may be symmetrically spaced about a bisecting line 2480 that bisects the distal end of the outer shaft 2409a. The configuration illustrated in FIG. 24A using the four slits 2455aa-2455ad may yield four phalanges of equal circumferential size relative to the circumference of the outer shaft 2409a. While the symmetrical configuration illustrated includes the slits 2455aa-2455ad being equally spaced apart, it will be appreciated that other symmetrically spaced configurations may not be equally spaced about the circumference of the outer shaft 2409a. For example, the first two slits 2455aa and 2455ab may be closer to each other than they are to the second two slits 2455ac and 2455ad.

As illustrated in FIG. 24B, the slits 2455ba-2455bd may be asymmetrically spaced about the bisecting line 2480 that bisects the distal end of the outer shaft 2409b. The configuration illustrated in FIG. 24B using the four slits 2455ba-2455bd may yield four phalanges of differing circumferential sizes relative to the circumference of the outer shaft 2409b. While one example configuration of asymmetrical distribution of the slits 2455ba-2455bd is illustrated, it will be appreciated that any asymmetrical configuration is contemplated.

As illustrated in FIG. 24C, the slits 2455ca-2455cc may be equally spaced about a circumference of the outer shaft 2409c. The configuration illustrated in FIG. 24C using the three slits 2455ca-2455cc may yield three phalanges of equal circumferential size relative to the circumference of the outer shaft 2409c.

As illustrated in FIG. 24D, the slits 2455da-2455df may be equally spaced about a circumference of the outer shaft 2409d. The configuration illustrated in FIG. 24D using the six slits 2455da-2455df may yield six phalanges of equal circumferential size relative to the circumference of the outer shaft 2409d.

In some embodiments, the flaring openings of the present disclosure may include any number of slits or openings. For example, the flaring openings may include between one and twelve slits or openings. As another example, the flaring openings may include one slit or opening. As another example, the flaring openings may include two slits or openings. As another example, the flaring openings may include three slits or openings. As another example, the flaring openings may include four slits or openings. As another example, the flaring openings may include between five and eight slits or openings. As another example, the flaring openings may include between nine and twelve slits or openings. Additionally or alternatively, the number of slits or openings may include any ranges therebetween and/or bounded by any numerical range or value identified herein.

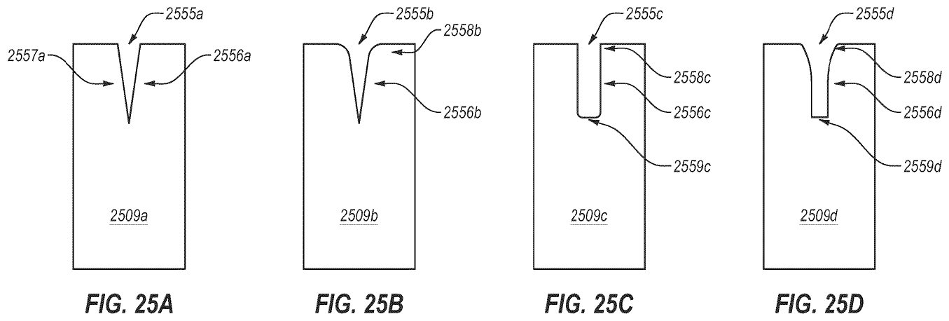

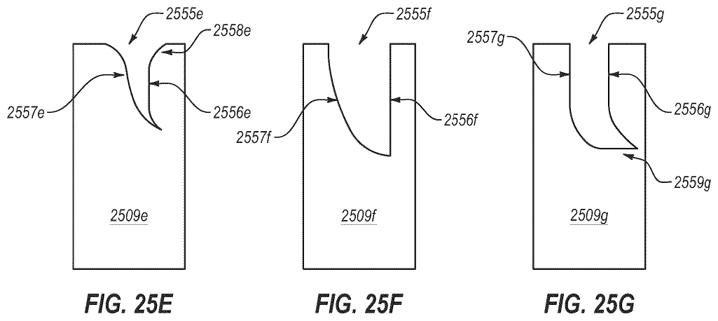

FIGS. 25A-25G illustrate front views of various embodiments of flaring openings 2555 in an outer shaft 2509. In the embodiments illustrated in FIGS. 25A-25G, a portion of the material of the outer shaft 2509 is removed to form the flaring openings 2555, rather than merely being a slit or a cut in the material of the outer shaft 2509.

The outer shafts 2509 may be similar or comparable to other outer shafts of the present disclosure, such as the outer shafts 2209, 2309, 2409, or 309. Additionally or alternatively, the flaring openings 2555 may be similar or comparable to other flaring openings, such as the flaring opening 2255, and/or the slits 2355, 2356, or 2455.

As illustrated in FIG. 25A, the flaring opening 2555a in the distal end of the outer shaft 2509a may include a wedge-shaped profile. The wedge-shape may include a first side 2556a and a second side 2557a that are each in distinct, generally straight lines that are intersecting to form the wedge-shape.

As illustrated in FIG. 25B, the flaring opening 2555b in the distal end of the outer shaft 2509b may include a generally wedge-shaped profile with a curved opening. The wedge-shape may include curved sides 2556b that include a first curved portion proximate the distal end of the outer shaft 2509b followed by a second portion that is generally straight until the two sides meet.

The curve of the sides 2556b form a shoulder 2558b that is rounded. In some embodiments, the rounded shoulder 2558b may make it easier for the implant to re-enter the outer shaft 2509b.

As illustrated in FIG. 25C, the flaring opening 2555c in the distal end of the outer shaft 2509c may include a generally rounded box-shaped profile. The flaring opening 2555c may include generally straight sides that follow the generally longitudinal direction of the outer shaft 2509c. The transition of the sides 2556c to the base 2559c of the flaring opening 2555c may include a curved profile. The shoulder 2558c may include a sharp corner rather than a rounded corner. While the sides 2556c are illustrated as parallel, the sides 2556c may be at angle such that they partially converge before reaching the base 2559c or partially converge before reaching the distal end of the outer shaft 2509c, all of which are contemplated by a box-shaped profile.

As illustrated in FIG. 25D, the flaring opening 2555d in the distal end of the outer shaft 2509d may include two curved sides 2556d leading to a base 2559d. The flaring opening 2555d may include rounded shoulders 2558d while the base 2559d is generally flat.

The embodiments illustrated in FIGS. 25A-25D are generally symmetrical in the longitudinal direction of the outer shaft 2509. The embodiments illustrated in FIGS. 25E-25G are asymmetrical about the longitudinal direction of the outer shaft 2509.

As illustrated in FIG. 25E, the flaring opening 2555e in the distal end of the outer shaft 2509e may include a wedge-shaped profile that curves to one side. The wedge-shaped profile that curves to one side may form rounded shoulders 2558e. In some embodiments, the first side 2556e may have a first curve profile (e.g., with a continuous curvature) and the second side 2557e may have a second curve profile (e.g., with a first portion curved in one direction and a second portion curved in an opposite direction).

As illustrated in FIG. 25F, the flaring opening 2555f in the distal end of the outer shaft 2509f may include a wedge-shaped profile that curves on one side. The wedge-shaped profile may include the first side 2556f that includes a generally straight line along the longitudinal direction of the outer shaft 2509f. The second side 2557f may include a curved profile that curves to meet the first side 2556f.

As illustrated in FIG. 25G, the flaring opening 2555g in the distal end of the outer shaft 2509g may include a profile that curves to one side. For example, the flaring opening 2555g may include two generally parallel sides 2556g and 2557g with a generally flat base 2559g.

While a single opening is illustrated in the embodiments of FIGS. 25A-25G, it will be appreciated that the flaring openings 2555 may include any number of flaring openings. For example, three similar openings may be spaced around the circumference of the outer shaft 2509. Additionally or alternatively, multiple different profiles or styles of openings may be included in a single embodiment. For example, two flaring openings may include the profile illustrated in FIG. 25B and two additional flaring openings may include the profile illustrated in FIG. 25E. In some embodiments, the flaring openings may include different lengths of openings. For example, a first flaring opening may be of a first length using the profile illustrated in FIG. 25A, a second flaring opening of a second length longer than the first flaring opening using the profile illustrated in FIG. 25A, and a third flaring opening of a third length longer than the second flaring opening using the profile illustrated in FIG. 25E.

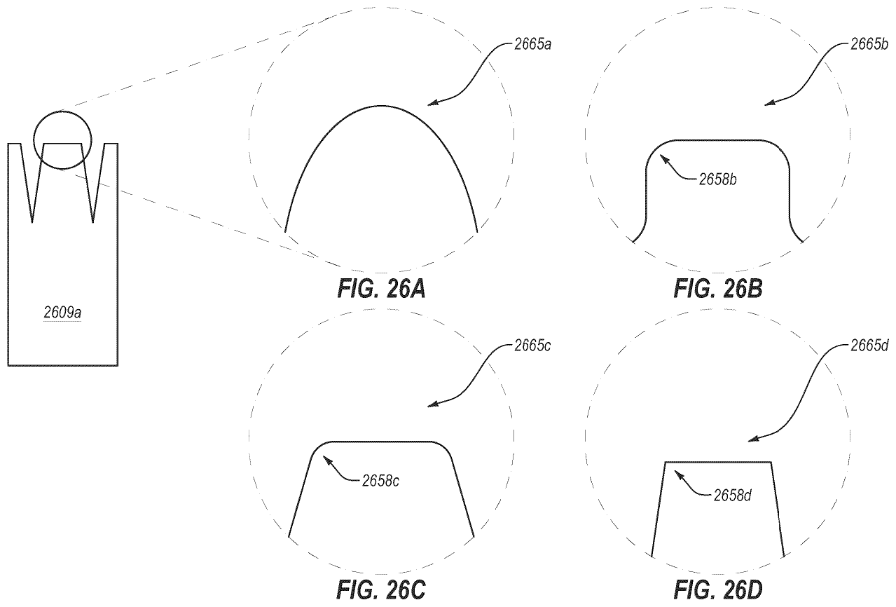

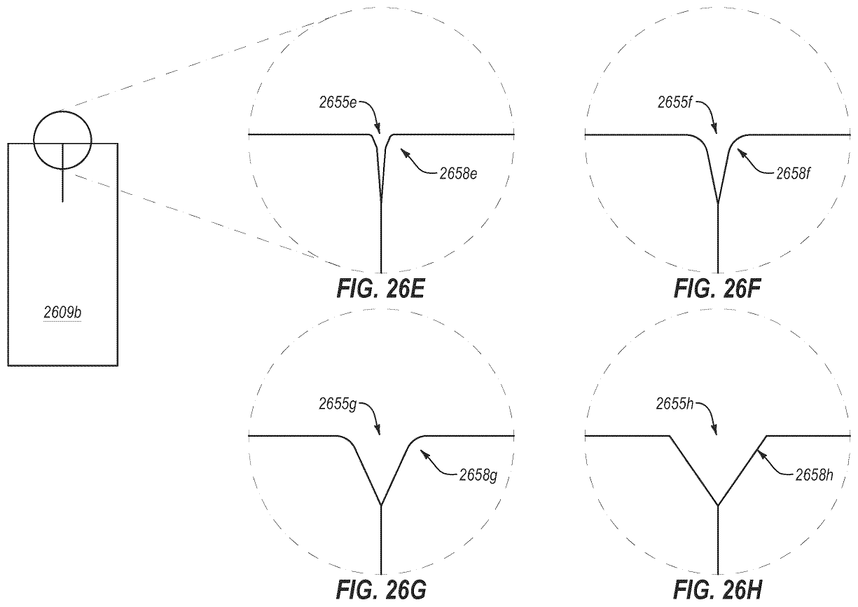

FIGS. 26A-26H illustrate front views of various profiles of phalanges 2665 created by flaring openings in an outer shaft or profiles of the flaring openings 2655 themselves at a distal end of an outer shaft 2609. FIGS. 26A-26D illustrate various profiles of the phalanges 2665 for an outer shaft 2609a, and FIGS. 26E-26H illustrate various profile of the flaring openings 2655 themselves for an outer shaft 2609b. The flaring openings 2655 may include a slit for a portion of the length and a portion removed to change the shoulders of the phalanges.

The outer shafts 2609 may be similar or comparable to other outer shafts of the present disclosure, such as the outer shafts 2209, 2309, 2409, 2509, or 309. Additionally or alternatively, the flaring openings 2655 may be similar or comparable to other flaring openings, such as the flaring opening 2255, 2555, and/or the slits 2355, 2356, or 2455. Additionally or alternatively, the phalanges 2665 may be similar or comparable to other phalanges such as the phalanges 2265 and/or 2365.

As illustrated in FIG. 26A, the phalange 2665a may include a curved profile, such as a parabolic profile. As illustrated in FIG. 26B, the phalange 2665b may include a profile with rounded shoulders 2658b and a rounded portion between phalanges and a generally flat portion at the distal end of the outer shaft 2609a. As illustrated in FIG. 26C, the phalange 2665c may include a profile with rounded shoulders 2658c that are less rounded than the shoulders 2658b of FIG. 26B, and with generally straight sides proceeding away from the distal end of the outer shaft 2609a. As illustrated in FIG. 26D, the phalange 2665d may include a profile with sharp shoulders 2658d and straight edges.

As illustrated in FIG. 26E, the flaring opening 2655e may include a slit generally, with a small portion removed at the distal end of the outer shaft 2609b. The shoulder 2658e may be slightly rounded. As illustrated in FIG. 26F, the flaring opening 2655f may include a slit generally, with a slightly larger portion removed at the distal end of the outer shaft 2609b. The shoulder 2658f may be more rounded than the shoulder 2658e. As illustrated in FIG. 26G, the flaring opening 2655g may include a slit generally, with an even larger portion removed at the distal end of the outer shaft 2609b. The shoulder 2658g may be more rounded than the shoulder 2658e. As illustrated in FIG. 26H, the flaring opening 2655h may include a slit generally, with portion removed at the distal end of the outer shaft 2609b using straight lines. The shoulder 2658h may include straight-edge corners, but due to the removal of the material the shoulder 2658h may have the effect of slightly rounded corners when facilitating the implant being retracted back within the outer shaft 2609b.

The various phalanges 2665 and flaring openings 2655 may be incorporated or applicable to any of the embodiments included in the present disclosure. For example, the slits illustrated in FIGS. 23A-23D and/or 24A-24D may include the profiles at the distal end of the outer shaft 2309 and/or 2409 as illustrated in FIGS. 26E-26H.

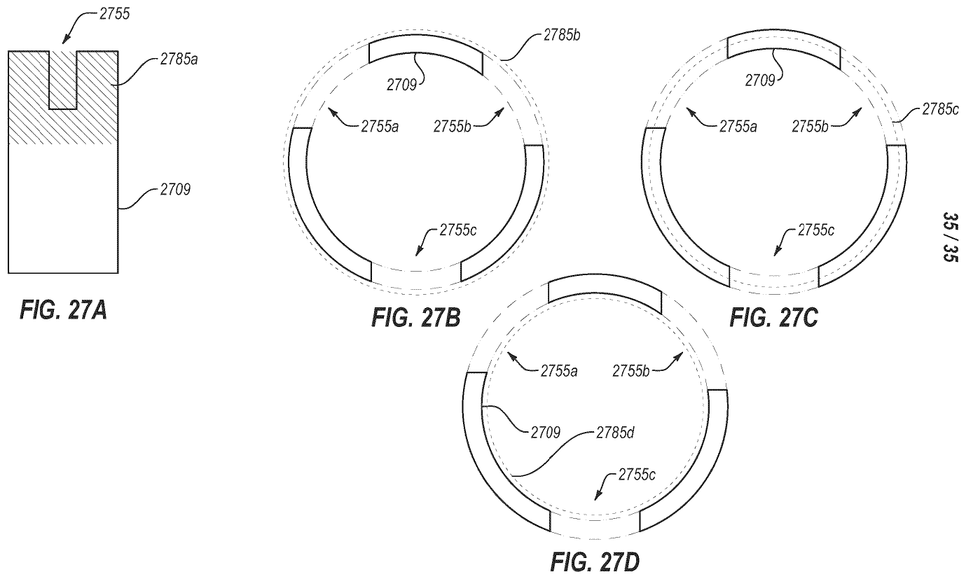

FIGS. 27A-27D illustrate various views of an outer shaft 2709 that includes webbing 2785, such as the webbing 2785a-2785d. The outer shafts 2709 may be similar or comparable to other outer shafts of the present disclosure, such as the outer shafts 2209, 2309, 2409, 2509, 2609, or 309. Additionally or alternatively, the flaring openings 2755 may be similar or comparable to other flaring openings, such as the flaring opening 2255, 2555, 2655 and/or the slits 2355, 2356, or 2455.

As illustrated in FIGS. 27A-27D, in some embodiments the outer shaft 2709 may include webbing 2785 associated with the flaring opening 2755. The webbing 2785 may include an elastic material such as rubber, nylon, thermoplastic polyurethane (TPU), or any other material with clastic properties. The webbing 2785 may provide support to maintain the phalanges in position and/or to prevent the implant from catching or otherwise getting caught in the flaring openings 2755. For example, the webbing 2785 may be a thin membrane attached at or otherwise formed with the distal end of the outer shaft 2709.

In some embodiments, the webbing 2785 may extend for any given distance along the length of the outer shaft 2709. For example, the webbing 2785 may extend for the entire length of the flaring opening 2755, for a distance beyond the length of the flaring opening 2755, or for less than the entire length of the flaring opening 2755. In some embodiments, the webbing 2785 may not reach all the way to the distal end of the outer shaft 2709, or stated another way, there may be a portion of the distal end of the outer shaft 2709 without the webbing 2785. In some embodiments, the webbing 2785 may be disposed in a band covering a portion of the flaring opening 2785 without reaching the distal end of the outer shaft 2709 and without reaching the end of the flaring opening 2785.

As illustrated in FIG. 27B, in some embodiments, the webbing 2785b may be disposed on the outside of the outer shaft 2709. For example, the outer shaft 2709 may include the flaring openings 2755a, 2755b, and 2755c with the webbing 2785b extending around the entire circumference of the outer shaft 2709. Stated another way, in some embodiments, the webbing 2785b may be disposed outside of an outer diameter of the outside shaft 2709.

As illustrated in FIG. 27C, in some embodiments, the webbing 2785c may be disposed at some point within the material of the outer shaft 2709. For example, the outer shaft 2709 may include the flaring openings 2755a, 2755b, and 2755c with the webbing 2785c extending within the outer shaft 2709 about the entire circumference of the outer shaft 2709. Stated another way, in some embodiments, the webbing 2785c may be disposed between an outer diameter and an inside diameter of the outside shaft 2709.

As illustrated in FIG. 27D, in some embodiments, the webbing 2785d may be disposed within the outer shaft 2709. For example, the outer shaft 2709 may include the flaring openings 2755a, 2755b, and 2755c with the webbing 2785d extending within the outer shaft 2709 about the entire circumference of the outer shaft 2709. Stated another way, in some embodiments, the webbing 2785d may be disposed inside of an inside diameter of the outside shaft 2709.

In some embodiments, the webbing 2785 may be disposed only within the flaring openings 2755a, 2755b, and 2755c and not about the entire circumference of the outer shaft 2709.

In some embodiments, the webbing 2785 may extend and/or cover at least a portion of the distal face of the outer shaft 2709 (e.g., the face viewed in FIGS. 27B-27D). In some embodiments, the webbing 2785b may be disposed on the outside of the outer shaft 2709 (such as illustrated in FIG. 27B), extend across the distal face, and extend for some distance along the inside of the outer shaft 2709 (such as illustrated in FIG. 27D).

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1: A delivery apparatus comprises a handle body having a proximal end, a distal end, a longitudinal axis extending between the proximal end and the distal end, and a cavity disposed between the proximal end and the distal end; a carriage member disposed within the cavity and axially movable relative to the handle body in a direction parallel to the longitudinal axis of the handle body, the carriage member having an internal surface defining an internal bore, a gland shoulder integrally formed with the internal surface and defining a stepdown transition in the internal bore; an outer shaft having a proximal end positioned within the internal bore of the carriage member and in opposing relation to the gland shoulder, wherein the proximal end of the outer shaft, the gland shoulder of the carriage member, and a portion of the internal surface of the carriage member adjacent to the stepdown transition define an annular groove; an inner shaft extending through a lumen of the outer shaft and fixed relative to the handle body; and a seal member disposed around the inner shaft and within the annular groove, wherein the seal member is positioned to form a seal between the carriage member and the inner shaft and at the proximal end of the outer shaft.

Example 2: The delivery apparatus of any example herein, particularly example 1, wherein the carriage member further comprises a locating shoulder integrally formed with the internal surface and axially displaced from the gland shoulder, and wherein the proximal end of the outer shaft abuts the locating shoulder of the carriage member to define the annular groove.

Example 3: The delivery apparatus of any example herein, particularly any one of examples 1 and 2, wherein the gland shoulder is an annular shoulder.

Example 4: The delivery apparatus of any example herein, particularly any one of examples 1 to 3, wherein the carriage member is a one-piece molded body.

Example 5: The delivery apparatus of any example herein, particularly any one of examples 1 to 4, wherein the outer shaft is coupled to the carriage member and movable relative to the handle body by the carriage member.

Example 6: The delivery apparatus of any example herein, particularly any one of examples 1 to 5, further comprises a first guide member formed in the carriage member; and a second guide member formed in the handle body; wherein the first and second guide members guide axial movement of the carriage member along the longitudinal axis and restrict relative rotational movement between the carriage member and the handle body.

Example 7: The delivery apparatus of any example herein, particularly any one of examples 1 to 5, further comprises opposed first and second longitudinal slots formed in the carriage member; and opposed first and second flattened projections formed on the handle body; wherein the opposed first and second flattened projections respectively extend into the opposed first and second longitudinal slots to guide axial movement of the carriage member along the longitudinal axis and inhibit relative rotational movement between the carriage member and the handle body.

Example 8: The delivery apparatus of any example herein, particularly any one of examples 1 to 7, wherein the carriage member comprises a head portion and a stem portion, and wherein the internal bore is formed in the head portion.

Example 9: The delivery apparatus of any example herein, particularly example 8, wherein the stem portion comprises a central opening that is connected to the internal bore, and wherein the internal bore and the central opening form a passage extending along a length of the carriage member.

Example 10: The delivery apparatus of any example herein, particularly example 9, wherein the head portion comprises an external threaded surface.

Example 11: The delivery apparatus of any example herein, particularly example 10, further comprises a drive member rotatably supported within the handle body, the drive member having an internal threaded surface that is threadedly engaged with the external threaded surface of the carriage member; wherein rotation of the drive member relative to the handle body results in the carriage member moving relative to the handle body along the longitudinal axis.

Example 12: The delivery apparatus of any example herein, particularly example 11, wherein the drive member comprises a barrel portion received partially within the cavity of the handle body and a knob portion operable to rotate the barrel portion relative to the handle body, and wherein the internal threaded surface is formed in the barrel portion and the knob portion.

Example 13: The delivery apparatus of any example herein, particularly any one of examples 1 to 12, further comprises an injection port disposed at the proximal end of the handle body, the injection port fluidly connected to a lumen of the inner shaft.

Example 14: A delivery assembly comprises the delivery apparatus of any example herein, particularly any one of examples 1 to 13, and an expandable docking station releasably coupled to the delivery apparatus, wherein the expandable docking station is configured to receive a prosthetic heart valve.

Example 15: A method comprises inserting a distal end of the delivery assembly of any example herein, particularly example 14, into a vasculature of a patient; advancing the distal end of the delivery assembly through the vasculature of the patient to position the expandable docking station at a select implantation location; and moving the carriage member relative to the handle to release the expandable docking station from the delivery apparatus.

Example 16: The method of any example herein, particularly example 15, wherein moving the carriage member relative to the handle body to release the expandable docking station from the delivery apparatus comprises moving the carriage member relative to the handle body to retract the outer shaft and expose the expandable docking station.

Example 17: The method of any example herein, particularly example 16, further comprises prior to inserting the distal end of the delivery assembly into the vasculature of the patient, moving the carriage member relative to the handle body to encapsulate the expandable docking station within the delivery apparatus.

Example 18: The method of any example herein, particularly example 17, wherein moving the carriage relative to the handle body to encapsulate the expandable docking station within the delivery apparatus comprises moving the carriage member relative to the handle body to extend the outer shaft over the expandable docking station.

Example 19: A handle for a prosthetic implant delivery apparatus comprises a handle body having a longitudinal axis and a cavity extending along the longitudinal axis; and a carriage member disposed within the cavity and axially movable relative to the longitudinal axis of the handle body, the carriage member comprising a carriage body having an internal surface defining an internal bore, a gland shoulder integrally formed with the internal surface and defining a stepdown transition in the internal bore; wherein the gland shoulder and a portion of the internal surface adjacent to the stepdown transition form a portion of an annular groove configured to receive a seal member.

Example 20: The handle of any example herein, particularly example 19, wherein the gland shoulder is an annular shoulder.

Example 21: The handle of any example herein, particularly example 19 or 20, wherein the carriage body further comprises a locating shoulder integrally formed with the internal surface and axially displaced from the gland shoulder, and wherein the portion of the annular groove is disposed between the gland shoulder and the locating shoulder.

Example 22: The handle of any example herein, particularly any one of examples 19 to 21, wherein the carriage body is a one-piece molded body.

Example 23: The handle of any example herein, particularly any one of examples 19 to 22, further comprises opposed first and second longitudinal slots formed in the carriage body; and opposed first and second flattened projections formed on the handle body; wherein the opposed first and second flattened projections respectively extend into the opposed first and second longitudinal slots to guide movement of the carriage member along the longitudinal axis of the handle body.

Example 24: The handle of any example herein, particularly any one of examples 19 to 23, wherein the carriage body comprises a head portion and a stem portion, and wherein the internal bore is formed in the head portion.

Example 25: The handle of any example herein, particularly example 24, wherein the stem portion comprises a central opening that is connected to the internal bore, and wherein the internal bore and the central opening form a passage extending along a length of the carriage body.

Example 26: The handle of any example herein, particularly any one of examples 24 and 25, wherein the head portion comprises an external threaded surface.

Example 27: The handle of any example herein, particularly example 26, further comprises a drive member rotatably supported within the handle body, the drive member having an internal threaded surface that is threadedly engaged with the external threaded surface; wherein rotation of the drive member relative to the handle body results in the carriage member moving relative to the handle body along the longitudinal axis.

Example 28: The handle of any example herein, particularly example 27, wherein the drive member comprises a barrel portion received partially within the cavity of the handle body and a knob portion operable to rotate the barrel portion relative to the handle body, and wherein the internal threaded surface is formed in the barrel portion and the knob portion.

Example 29: The handle of any example herein, particularly any one of examples 19 to 25, further comprising a drive member operatively coupled to the carriage member to move the carriage member relative to the handle body.

Example 30: A carriage for a prosthetic implant delivery apparatus comprises a one-piece molded body having an internal surface defining an internal bore, a gland shoulder integrally formed with the internal surface and defining a stepdown transition in the internal bore; wherein the gland shoulder and a portion of the internal surface adjacent to the stepdown transition form a portion of an annular groove configured for receiving a seal member.

Example 31: The carriage of any example herein, particularly example 30, wherein the gland shoulder is an annular shoulder.

Example 32: The carriage of any example herein, particularly any one of examples 30 and 31, wherein the one-piece molded body further comprises a locating shoulder integrally formed with the internal surface and axially displaced from the gland shoulder, and wherein the portion of the annular groove is disposed between the gland shoulder and the locating shoulder.

Example 33: The carriage of any example herein, particularly any one of examples 30 and 32, wherein the one-piece molded body comprises a head portion and a stem portion, and wherein the internal bore is formed in the head portion.

Example 34: The carriage of any example herein, particularly example 33, wherein the stem portion comprises a central opening that is connected to the internal bore, and wherein the internal bore and the central opening form a passage extending along a length of the one-piece molded body.

Example 35: The carriage of any example herein, particularly any one of examples 33 and 34, wherein the head portion comprises an external threaded surface.

Example 36: The carriage of any example herein, particularly any one of examples 34 and 35, further comprising opposed first and second longitudinal slots formed at least partially in the stem portion and extending parallel to the passage.

Example 37: The carriage of any example herein, particularly example 36, wherein at least one of the first and second longitudinal slots is connected to the passage.

Example 38: A method of forming a component of a prosthetic implant delivery apparatus comprises securing a core pin within a mold cavity; and injecting a thermoplastic material into the mold cavity to form a molded body having an internal surface defining an internal bore, a gland shoulder integrally formed with the internal surface and defining a stepdown transition in the internal bore.

Example 39: A method of forming a component of a prosthetic implant delivery apparatus comprises securing a core pin within a mold cavity; and injecting a thermoplastic material into the mold cavity to form a molded body having an internal surface defining an internal bore, a gland shoulder integrally formed with the internal surface and defining a stepdown transition shoulder in the internal bore, and a locating shoulder integrally formed with the internal surface and axially displaced from the gland shoulder.

Example 40: A delivery apparatus comprises a handle body having a longitudinal axis and a cavity extending along the longitudinal axis; an outer shaft having a proximal end positioned within the cavity, the outer shaft having a first lumen; an inner shaft extending through the first lumen of the outer shaft, the inner shaft having a second lumen and one or more fluid ports that fluidly connect the second lumen to the first lumen; and an injection port fluidly connected to the second lumen of the inner shaft, wherein both the first and second lumens are flushable with a fluid through the injection port.

Example 41: The delivery apparatus of any example herein, particularly example 40, wherein the inner shaft comprises a reinforced tube, and wherein the one or more fluid ports are formed in a wall of the reinforced tube.

Example 42: The delivery apparatus of any example herein, particularly example 41, wherein the reinforced tube comprises an inner layer, a reinforcement layer disposed over the inner layer, and an outer layer disposed over the reinforcement layer, and wherein the second lumen is formed within the inner layer.

Example 43: The delivery apparatus of any example herein, particularly example 42, wherein the reinforcement layer comprises a braided material.

Example 44: The delivery apparatus of any example herein, particularly example 43, wherein each of the fluid ports comprises a first opening in the inner layer, a second opening in the outer layer that is radially aligned with the first opening, and a portion of the braided tube disposed between the first opening and the second opening.

Example 45: The delivery apparatus of any example herein, particularly any one of examples 41 to 44, wherein the one or more fluid ports are longitudinally aligned on the reinforced tube.

Example 46: The delivery apparatus of any example herein, particularly any one of examples 41 to 44, wherein the one or more fluid ports form a circular pattern around the reinforced tube.

Example 47: The delivery apparatus of any example herein, particularly any one of examples 41 to 44, wherein the one or more fluid ports form a spiral pattern around the reinforced tube.

Example 48: The delivery apparatus of any example herein, particularly any one of examples 41 to 47, further comprises a cover tube disposed over at least a portion of the reinforced tube, the cover tube having one or more windows positioned to expose the one or more fluid ports.

Example 49: The delivery apparatus of any example herein, particularly any one of examples 40 to 48, wherein the one or more fluid ports are formed in a portion of the inner shaft proximate to the handle body.

Example 50: The delivery apparatus of any example herein, particularly any one of examples 40 to 49, further comprises a carriage member disposed within the elongated cavity and movable relative to the handle body along the longitudinal axis of the handle body, the carriage member comprising a carriage body having a passage defined therein, wherein the proximal end of the outer shaft is positioned within the passage.

Example 51: The delivery apparatus of any example herein, particularly of example 50, further comprises a seal member disposed within the passage, wherein the seal member is positioned to form a seal at the proximal end of the outer shaft.

Example 52: A delivery assembly comprises the delivery apparatus of any example herein, particularly any one of examples 40 to 51, and an expandable docking station for an expandable valve releasably coupled to the delivery apparatus.

Example 53: A method comprises inserting a distal end of the delivery assembly of any example herein, particularly example 52, into a vasculature of a patient; advancing the delivery assembly through the vasculature of the patient to position the expandable docking station at a select implantation location; and moving the carriage member relative to the handle to release the expandable docking station from the delivery apparatus.

Example 54: A method comprises providing a reinforced tube comprising an inner layer, a reinforcement layer disposed over the inner layer, and an outer layer disposed over the reinforcement layer; and ablating the reinforced tube at one or more locations to form one or more fluid ports in the reinforced tube.

Example 55: A method comprises disposing a cover tube having one or more windows over a reinforced tube; and ablating the reinforced tube at one or more locations exposed through the one or more windows to form one or more fluid ports in the reinforced tube.

Example 56: The method of any example herein, particularly example 55, wherein the reinforced tube comprises an inner layer, a reinforcement layer disposed over the inner layer, and an outer layer disposed over the inner layer, and wherein ablating the reinforced tube comprises ablating the inner layer and the outer layer without ablating the reinforcement layer.

Example 57: A shaft assembly for a prosthetic implant delivery apparatus comprises an outer shaft having a first lumen and an inner shaft extending through the first lumen. The inner shaft comprises a reinforced tube having a second lumen and one or more fluid ports that fluidly connect the second lumen to the first lumen. The inner shaft further comprises a cover tube disposed over the reinforced tube, the cover tube having one or more windows positioned to expose the one or more fluid ports to the first lumen.

Example 58: The shaft assembly of any example herein, particularly example 57, wherein the reinforced tube comprises an inner layer, a reinforcement layer disposed over the inner layer, and an outer layer disposed over the reinforcement layer, and wherein the second lumen is formed within the inner layer.

Example 59: The shaft assembly of any example herein, particularly example 58, wherein the reinforcement layer comprises a braided material.

Example 60: The shaft assembly of any example herein, particularly example 59, wherein each of the fluid ports comprises a first opening in the inner layer, a second opening in the outer layer that is radially aligned with the first opening, and a portion of the braided material disposed between the first opening and the second opening.

Example 61: A delivery apparatus comprises an elongate shaft having a proximal end portion and a distal end portion, wherein the proximal end portion is configured to be disposed outside of a patient's body during a delivery procedure, and wherein the distal end portion is configured to be disposed inside a patient's body during the delivery procedure; and a frame connector coupled to the distal end portion of the elongate shaft and configured for releasably coupling a prosthetic implant to the delivery apparatus, the frame connector comprising a connector body having an exterior with an exterior surface and a recess, the recess comprising a first slot portion having a first width, a second slot portion having a second width that is larger than the first width, a recess floor, and opposed first and second side walls extending from the recess floor to the exterior surface and connected to the first slot portion and the second slot portion, wherein at least a first portion of each of the first and second side walls connected to the second slot portion includes an undercut from the exterior surface to the recess floor.

Example 62: The delivery apparatus of any example herein, particularly example 61, wherein the recess floor is recessed relative to the exterior surface of the connector body, and wherein the first and second side walls project from opposite sides of the recess floor.

Example 63: The delivery apparatus of any example herein, particularly example 62, wherein at least a first portion of each of the first and second side walls of the recess is inclined at an angle relative to the recess floor.

Example 64: The delivery apparatus of any example herein, particularly example 63, wherein the angle is within a range of 75-89.9 degrees.

Example 65: The delivery apparatus of any example herein, particularly example 63, wherein the angle is within a range of 81-86 degrees.

Example 66: The delivery apparatus of any example herein, particularly any one of examples 62 to 65, wherein a second portion of each of the first and second side walls connected to the first slot portion of the recess includes an undercut.

Example 67: The delivery apparatus of any example herein, particularly any one of examples 62 to 66, wherein the recess comprises an end wall that is longitudinally displaced from the first and second side walls of the recess, and wherein the end wall is connected to the second slot portion.

Example 68: The delivery apparatus of any example herein, particularly example 67, wherein a height of each of the first and second side walls of the recess relative to the recess floor is greater than a height of the end wall of the recess relative to the recess floor.

Example 69: The delivery apparatus of any example herein, particularly any one of examples 61 to 68, wherein each of the first and second side walls of the recess comprises a rounded corner where the first slot portion and the second slot portion of the recess are connected.

Example 70: The delivery apparatus of any example herein, particularly any one of examples 61 to 69, wherein the recess is one recess of a plurality of recesses, wherein a plurality of the recesses is formed in angularly spaced apart locations on the exterior of the connector body.

Example 71: The delivery apparatus of any example herein, particularly any one of examples 61 to 70, wherein the connector body comprises an internal bore, and wherein the elongate shaft extends through the internal bore.

Example 72: The delivery apparatus of any example herein, particularly any one of examples 61 to 71, wherein the frame connector comprises a flange formed at an end of the connector body, the flange comprising a plurality of radial holes.

Example 73: The delivery apparatus of any example herein, particularly example 72, wherein a portion of the elongate shaft extends over the flange and through the radial holes.

Example 74: The delivery apparatus of any example herein, particularly any one of examples 61 to 73, wherein the recess is open at the exterior surface.

Example 75: A frame connector for a prosthetic implant delivery apparatus comprises a connector body having an exterior with an exterior surface, a recessed surface, and opposed first and second side walls, the recessed surface spaced radially inwardly relative to the exterior surface and comprising a first slot portion having a first width and a second slot portion having a second width that is larger than the first width, the opposed first and second side walls extending radially from the recessed surface to the exterior surface and connected to the first slot portion and the second slot portion, wherein at least a first portion of each of the first and second side walls connected to the second slot portion forms an angle relative to the recessed surface within a range of 75-89.9 degrees.

Example 76: The frame connector of any example herein, particularly example 75, wherein the angle is within a range of 81-86 degrees.

Example 77: The frame connector of any example herein, particularly any one of examples 75 and 76, wherein a second portion of each of the first and second side walls connected to the first slot portion forms an angle relative to the recessed surface within a range of 75-89.9 degrees.

Example 78: The frame connector of any example herein, particularly any one of examples 75 to 77, wherein each of the first and second side walls comprises an undercut extending along an entire length of the respective first and second side walls.

Example 79: The frame connector of any example herein, particularly any one of examples 75 to 78, wherein each of the first and second side walls comprises a rounded corner where the first slot portion and the second slot portion are connected.

Example 80: The frame connector of any example herein, particularly any one of examples 75 to 79, wherein the recessed surface is one of a plurality of the recessed surfaces formed in angularly spaced apart locations on the exterior of the connector body.

Example 81: The frame connector of any example herein, particularly any one of examples 75 to 80, wherein the connector body comprises an internal bore.

Example 82: The frame connector of any example herein, particularly any one of examples 75 to 81, further comprises a flange formed at an end of the connector body, the flange comprising a plurality of radial holes.

Example 83: A delivery assembly comprises a self-expandable docking station comprising at least one connector tab, the at least one connector tab having a flared portion; and a frame connector comprising a connector body having at least one recess to receive and retain the at least one connector tab, the at least one recess comprising a slot portion to receive the flared portion, a recess floor, and opposed first and second side walls connected to the slot portion and the recess floor, wherein at least a portion of each of the first and second side walls forms an angle relative to the recess floor within a range of 75-89.9 degrees.

Example 84: A shaft assembly to facilitate delivery of an implant to an implantation location within a patient's anatomy. The shaft assembly may comprise an outer shaft that is movable between an extended position to encapsulate the implant loaded onto a delivery apparatus and a retracted position to expose the implant for deployment at the implantation location at a distal end of the outer shaft, the outer shaft movable from the retracted position to the extended position to encapsulate the implant at least partially within the distal end of the outer shaft, the outer shaft comprising one or more flaring openings at the distal end of the outer shaft.

Example 85: A delivery assembly may comprise a delivery apparatus that comprises a handle body having a proximal end, a distal end, a longitudinal axis extending between the proximal end and the distal end, and a cavity disposed between the proximal end and the distal end. The delivery apparatus may further comprise a carriage member disposed within the cavity and axially movable relative to the handle body in a direction parallel to the longitudinal axis of the handle body, the carriage member having an internal surface defining an internal bore. The delivery apparatus may also comprise an outer shaft having a proximal end positioned within the internal bore of the carriage member and one or more flaring openings at a distal end of the outer shaft. The delivery apparatus may additionally comprise an inner shaft extending through a lumen of the outer shaft and fixed relative to the handle body. The delivery assembly may also comprise an implant removably loaded onto the delivery apparatus.

Example 86: The shaft assembly and/or delivery assembly of any example herein, particularly any one of examples 84-85, wherein the flaring openings include one or more slits originating at the distal end of the outer shaft.

Example 87: The shaft assembly and/or delivery assembly of any example herein, particularly example 86, wherein the flaring openings include between three and six slits.

Example 88: The shaft assembly and/or delivery assembly of any example herein, particularly examples 86-87, wherein the one or more slits include a first slit of a first length and a second slit of a second length different from the first length.

Example 89: The shaft assembly and/or delivery assembly of any example herein, particularly examples 86-88, wherein at least one of the one or more slits progresses in a generally longitudinal direction relative to the outer shaft when extending away from the distal end of the outer shaft.

Example 90: The shaft assembly and/or delivery assembly of any example herein, particularly examples 86-88, wherein at least one of the one or more slits progresses in both a longitudinal direction and a circumferential direction relative to the outer shaft when extending away from the distal end of the outer shaft.

Example 91: The shaft assembly and/or delivery assembly of any example herein, particularly examples 84-85, wherein the flaring openings include a wedge-shaped opening.

Example 92: The shaft assembly and/or delivery assembly of any example herein, particularly example 91, wherein at least one side of the wedge-shaped opening is curved.

Example 93: The shaft assembly and/or delivery assembly of any example herein, particularly examples 84-85, wherein the flaring openings include a box-shaped opening.

Example 94: The shaft assembly and/or delivery assembly of any example herein, particularly example 93, wherein at least one side of the box-shaped opening is curved.

Example 95: The shaft assembly and/or delivery assembly of any example herein, particularly examples 93-94, wherein at least one side of the box-shaped opening is angled inward such that as the box-shaped opening progresses in a longitudinal direction along the outer shaft and originating at the distal end of the outer shaft, the box-shaped opening becomes more narrow.

Example 96: The shaft assembly and/or delivery assembly of any example herein, particularly examples 84-85, wherein the flaring openings include at least one slit and at least one wedge-shaped opening.

Example 97: The shaft assembly and/or delivery assembly of any example herein, particularly examples 84-85, wherein the flaring openings include at least one slit and at least one box-shaped opening.

Example 98: The shaft assembly and/or delivery assembly of any example herein, particularly examples 84-85, wherein the flaring openings include at least one wedge-shaped opening and at least one box-shaped opening.

Example 99: The shaft assembly and/or delivery assembly of any example herein, particularly examples 84-85, wherein the flaring openings include at least one slit, at least one wedge-shaped opening, and at least one box-shaped opening.

Example 100: The shaft assembly and/or delivery assembly of any example herein, particularly examples 84-99, wherein the flaring openings extend between 0.1× of an outer diameter of the outer shaft and 2× of the outer diameter of the outer shaft.

Example 101: The shaft assembly and/or delivery assembly of any example herein, particularly example 100, wherein at least one of the flaring openings extends between 0.5× of the outer diameter of the outer shaft and 1× of the outer diameter of the outer shaft.

Example 102: The shaft assembly and/or delivery assembly of any example herein, particularly examples 84-101, wherein the flaring openings include a plurality of openings that are spaced symmetrically about a bisecting line of a circumference of the outer shaft.

Example 103: The shaft assembly and/or delivery assembly of any example herein, particularly examples 84-101, wherein the flaring openings include a plurality of openings that are spaced asymmetrically about a bisecting line of a circumference of the outer shaft.

Example 104: The shaft assembly and/or delivery assembly of any example herein, particularly examples 84-101, wherein the flaring openings include a plurality of openings that are evenly spaced about a circumference of the outer shaft.

Example 105: The shaft assembly and/or delivery assembly of any example herein, particularly examples 84-104, wherein the flaring openings are configured to permit outward displacement of at least a portion of an outer circumference of the distal end of the outer shaft as the outer shaft is at least partially extended over the implant.

Example 106: The shaft assembly and/or delivery assembly of any example herein, particularly example 105, wherein the portion of the outer circumference of the distal end of the outer shaft is configured to be outwardly displaced by interference of the implant with an inner surface of the outer shaft proximate the distal end of the outer shaft, forcing the portion radially outwards.

Example 107: The shaft assembly and/or delivery assembly of any example herein, particularly examples 105-106, wherein a geometry of the outer shaft permits deformation to accommodate the outward displacement of the portion of the outer circumference of the outer shaft.

Example 108: The shaft assembly and/or delivery assembly of any example herein, particularly examples 105-107, wherein a material of the outer shaft elastically deforms to accommodate the outward displacement of the portion of the outer circumference of the outer shaft.

Example 109: The shaft assembly and/or delivery assembly of any example herein, particularly examples 84-108, wherein the implant includes a stent.

Example 110: The shaft assembly and/or delivery assembly of any example herein, particularly examples 84-108, wherein the implant includes an expandable docking station.

Example 111: The shaft assembly and/or delivery assembly of any example herein, particularly example 110, wherein the expandable docking station is configured to receive a prosthetic heart valve.

Example 112: The shaft assembly and/or delivery assembly of any example herein, particularly example 85, wherein the outer shaft is movable between an extended position to at least partially encapsulate the implant and a retracted position to expose the implant for deployment at an implantation location at a distal end of the outer shaft.

Example 113: The shaft assembly and/or delivery assembly of any example herein, particularly example 112, wherein the flaring openings are configured to permit outward displacement of at least a portion of an outer circumference of the distal end of the outer shaft as the outer shaft is extended to at least partially encapsulate the implant within the outer shaft.

Example 114: The shaft assembly and/or delivery assembly of any example herein, particularly examples 84-113, further comprising webbing about the outer shaft at the distal end of the outer shaft.

Example 115: The shaft assembly and/or delivery assembly of any example herein, particularly example 114, wherein the webbing is disposed outside of the outside shaft.

Example 116: The shaft assembly and/or delivery assembly of any example herein, particularly example 114, wherein the webbing is disposed between an outer diameter and an inner diameter of the outside shaft.

Example 117: The shaft assembly and/or delivery assembly of any example herein, particularly example 114, wherein the webbing is disposed inside of the outside shaft.

Example 118: A method may comprise inserting a distal end of the shaft assembly and/or delivery assembly of any example herein, particularly example 84-85, into a vasculature of a patient. The method may also comprise advancing the delivery assembly through the vasculature of the patient to position the implant at a select implantation location. The method may additionally comprise moving the carriage member relative to the handle in a first direction to retract the outer shaft and expose the implant. The method may also comprise moving the carriage member relative to the handle in a second direction different from the first direction to extend the outer shaft to at least partially encapsulate the implant within the outer shaft.

Example 119: The method of any of the example methods herein, particularly example 118, wherein the flaring openings facilitate outward displacement of at least a portion of an outer circumference of the distal end of the outer shaft as the outer shaft is extended to at least partially encapsulate the implant within the outer shaft.

The features described herein with regard to any example can be combined with other features described in any one or more of the other examples, unless otherwise stated.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

What is claimed is:

1. A shaft assembly to facilitate delivery of an implant to an implantation location within a patient's anatomy, the shaft assembly comprising:
an outer shaft that is movable between an extended position to encapsulate the implant loaded onto a delivery apparatus and a retracted position to expose the implant for deployment at the implantation location at a distal end of the outer shaft, the outer shaft movable from the retracted position to the extended position to encapsulate the implant at least partially within the distal end of the outer shaft, the outer shaft comprising one or more flaring openings at the distal end of the outer shaft.

2. The shaft assembly of claim 1, wherein the flaring openings include one or more slits originating at the distal end of the outer shaft.

3. The shaft assembly of claim 2, wherein the flaring openings include between three and six slits.

4. The shaft assembly of claim 2, wherein the one or more slits include a first slit of a first length and a second slit of a second length different from the first length.

5. The shaft assembly of claim 2, wherein at least one of the one or more slits progresses in a generally longitudinal direction relative to the outer shaft when extending away from the distal end of the outer shaft.

6. The shaft assembly of claim 2, wherein at least one of the one or more slits progresses in both a longitudinal direction and a circumferential direction relative to the outer shaft when extending away from the distal end of the outer shaft.

7. The shaft assembly of claim 1, wherein the flaring openings include a wedge-shaped opening.

8. The shaft assembly of claim 7, wherein at least one side of the wedge-shaped opening is curved.

9. The shaft assembly of claim 1, wherein the flaring openings include a box-shaped opening.

10. The shaft assembly of claim 9, wherein at least one side of the box-shaped opening is curved.

11. The shaft assembly of claim 9, wherein at least one side of the box-shaped opening is angled inward such that as the box-shaped opening progresses in a longitudinal direction along the outer shaft and originating at the distal end of the outer shaft, the box-shaped opening becomes more narrow.

12. The shaft assembly of claim 1, wherein the flaring openings include at least one slit and at least one wedge-shaped opening.

13. The shaft assembly of claim 1, wherein the flaring openings include at least one slit and at least one box-shaped opening.

14. The shaft assembly of claim 1, wherein the flaring openings include at least one wedge-shaped opening and at least one box-shaped opening.

15. The shaft assembly of claim 1, wherein the flaring openings include at least one slit, at least one wedge-shaped opening, and at least one box-shaped opening.

16. A delivery assembly comprising:
a delivery apparatus comprising:
a handle body having a proximal end, a distal end, a longitudinal axis extending between the proximal end and the distal end, and a cavity disposed between the proximal end and the distal end;
a carriage member disposed within the cavity and axially movable relative to the handle body in a direction parallel to the longitudinal axis of the handle body, the carriage member having an internal surface defining an internal bore;
an outer shaft having a proximal end positioned within the internal bore of the carriage member and one or more flaring openings at a distal end of the outer shaft; and
an inner shaft extending through a lumen of the outer shaft and fixed relative to the handle body; and
an implant removably loaded onto the delivery apparatus.

17. The delivery assembly of claim 16, wherein the outer shaft is movable between an extended position to at least partially encapsulate the implant and a retracted position to expose the implant for deployment at an implantation location at a distal end of the outer shaft.

18. The delivery assembly of claim 17, wherein the flaring openings are configured to permit outward displacement of at least a portion of an outer circumference of the distal end of the outer shaft as the outer shaft is extended to at least partially encapsulate the implant within the outer shaft.

19. A method, comprising:
providing a delivery apparatus comprising
a handle body having a proximal end, a distal end, a longitudinal axis extending between the proximal end and the distal end, and a cavity disposed between the proximal end and the distal end,
a carriage member disposed within the cavity and axially movable relative to the handle body in a direction parallel to the longitudinal axis of the handle body, the carriage member having an internal surface defining an internal bore,
an outer shaft having a proximal end positioned within the internal bore of the carriage member and one or more flaring openings at a distal end of the outer shaft,
an inner shaft extending through a lumen of the outer shaft and fixed relative to the handle body, and
an implant removably loaded onto the delivery apparatus;
inserting a distal end of the delivery assembly into a vasculature of a patient;
advancing the delivery assembly through the vasculature of the patient to position the implant at a select implantation location;
moving the carriage member relative to the handle in a first direction to retract the outer shaft and expose the implant; and
moving the carriage member relative to the handle in a second direction different from the first direction to extend the outer shaft to at least partially encapsulate the implant within the outer shaft.

20. The method of claim 19, wherein the flaring openings facilitate outward displacement of at least a portion of an outer circumference of the distal end of the outer shaft as the outer shaft is extended to at least partially encapsulate the implant within the outer shaft.
